(19) United States
(12) Patent Application Publication
(10) Pub. No.: US 2024/0055134 A1
(43) Pub. Date: Feb. 15, 2024

(54) SYSTEMS AND METHODS FOR PROVIDING REAL-TIME ACCESS, QUEUE AND RISK MANAGEMENT (AQRM)

(71) Applicant: QFIRST SYSTEMS, INC., Park City, UT (US)

(72) Inventors: Kevin Maher, Park City, UT (US); Dan Oakeson, West Jordan, UT (US); Doug Reynolds, Clinton, UT (US); Davin Saderholm, Salt Lake City, UT (US)

(73) Assignee: QFIRST SYSTEMS, INC., Park City, UT (US)

(21) Appl. No.: 18/268,016

(22) PCT Filed: Dec. 10, 2021
(86) PCT No.: PCT/US2021/062943

§ 371 Date: Jun. 16, 2023

Related U.S. Application Data
(60) Provisional application No. 63/127,807, filed on Dec. 18, 2020.

(51) Int. Cl. G16H 50/30

(52) U.S. Cl. CPC G16H 50/30

(57) ABSTRACT

According to one aspect, a method for providing real-time access, queue, and risk management information to a user is provided. The method includes receiving, from a user device, a request indicating a date and time a user intends to visit a first establishment. The method includes determining a predicted occupancy of the first establishment for the date and time indicated in the request using a predicted occupancy model (POM). The method includes determining an exposure risk for the first establishment for the date and time indicated in the request using an exposure risk model (ERM). The method includes transmitting, to the user device, the predicted occupancy and the exposure risk for the first establishment. The method includes receiving, from the user device, a confirmation that the user will visit the first establishment at the date and time.

### TECHNICAL FIELD

Aspects of the present disclosure relate to Access, Queue, and Risk Management (AQRM) systems and methods utilizing an array of electronic devices, cloud resources, risk profiles of both Users and Establishments, and computational algorithms to collect, manage, and assess data for use in providing Users and Establishments with risk assessment information.

### BACKGROUND

The CoVID-19 pandemic has created many new challenges for patrons and Establishments who have been forced to adjust their operating practices and reduce capacity to meet required social distancing. Many Establishments, defined as a single physical location where a business, private organization, an entertainment or sporting venue, or a public institution conducts activities, provide services or perform operations involving interactions with the public. This also applies to those who control and actively operate the Establishment and lack the resources to actively manage their occupancy or to effectively communicate their status to patrons. Likewise, patrons now carry a dual burden of trying to assess the exposure risk, defined as, the risk one assumes while entering an Establishment where exposure to a communicable disease (e.g. Coronavirus) prevalent in their community is possible, and dealing with the potential inconvenience of waiting in a queue to enter an Establishment.

The CoVID-19 pandemic era has challenged many Establishments and forced them to adjust their operating practices to actively manage ingress/egress flow and the spacing of patrons to meet social distancing requirements. Many lack the resources or finances to support active access management nor do they have the means to effectively communicate their status to patrons.

Establishments face two extremes a) over-crowding, requiring outside queues and labor to manage lines, or worse, b) under-utilization as patrons opt to stay away even though the Establishment may be operating well below restricted capacity defined as, reduced occupancy levels driven by public health mandates to provide required social distancing. (Often presented in public health advisories as a % posted occupancy).

Patrons, or Users, are concerned about their risk of exposure to Coronavirus, or other communicable diseases prevalent in the community, by visiting crowded Establishments. They are also wary of wasting their time traveling to a destination without knowing about the crowd situation at the Establishment prior to their intended visit.

Line queuing has become a way of life for many waiting to enter Establishments operating at restricted occupancy levels. Inclement weather and poor queue management make this condition even less desirable.

### SUMMARY

Both Establishments and Users need a means to improve the flow of information and to facilitate the logistics of interaction. Establishments need to manage occupancy efficiently while patrons need accurate up-to-date information to make effective decisions regarding personal risk. Both are required to create or maintain trusted relationships and focused engagements between patrons and the Establishments they frequent.

According to some aspects, an Access, Queue, and Risk Management system (AQRM), gives both stakeholders the ability to manage on-site risk and occupancy, to establish appointment setting communications, to enable on-site social/physical distancing protocols, and to easily communicate incentives for visiting Establishments (businesses, locations, etc.). AQRM utilizes devices and other means set up within an Establishment to detect and report occupancy information to cloud resources, (remote servers for data storage and processing). This data is combined with latest public health advisories issued by government, defined as, a local, county, state or federal agency or department responsible for issuing and enforcing public health guidelines and/or restrictions, and future occupancy predictions from User inputs and past activity to provide a clear picture of occupancy conditions now and in the near future, so that Users can make informed decisions regarding their potential interaction.

According to some aspects, an end-to-end solution to measure and communicate occupancy information and risk projections to both Users and Establishments in real-time is provided. A device(s), or other means, at member Establishments detect personal electronic identifiers (PEI), defined as, a device, such as a mobile phone, BLE card or other electronic device that emits a radio (or other) signal containing a unique identifier that can be detected and read by other devices which continuously reports occupancy data to cloud computing resources. A Mobile and web-based APP, defined as, software that enables Users and Establishments to interact with the AQRM system, provides a simple Graphical User Interface (GUI) to both Users, defined as, a person, in the physical form, that utilizes the AQRM system to manage their interactions with Establishments throughout the course of their daily activities, and Establishments to facilitate the presentation of information in near real-time, and the transmission of their resulting decisions to the cloud.

According to some aspects, algorithm(s) may be employed that predict near-term and future occupancy based on User reservations, defined as, an act to designate and commit to a place in the current queue for an Establishment (same day—within 3 hours of current time), and appointments, defined as, the designation of a specific date and time to visit an Establishment (greater than 3 hours ahead of current time), in conjunction with ad-hoc traffic and analysis of past occupancy trends. Data analytics are delivered to Establishments for future resource planning. Other algorithms predict an exposure risk metric based upon the duration of interaction, prevalence of a communicable disease, such as Coronavirus, or other risks in the community, the type of Establishment and the occupancy levels along with other factors.

According to some aspects, the system allows Establishments to manage their ingress/egress activity and occupancy autonomously according to the parameters they select and in conjunction with current government restrictions, which can then be displayed or communicated at the Establishment via visual or audible means. It also enables Establishments(s) to plan resources for future occupancy.

According to some aspects, a mobile and web APP enables Users to assess exposure risk, occupancy, and social distancing to make informed decisions while planning visits, making reservations, or scheduling appointments to Establishments. Users are alerted if changing conditions exceed their pre-established exposure risk threshold based on a cumulative risk assessment score prior to and while visiting an Establishment.

According to some aspects, all permissions may be controlled by the User(s) on an opt-in basis (the default is 100% destroy/erase of personal data after each visit) providing high level of privacy. Users have sole control to enable mutually beneficial relationship(s) with their favorite or most used Establishments. They can elect to share personal preferences and data with each Establishment they designate as “trusted”. For example, Users can share vaccine or other health-related information or other permission-based information they desire with “trusted” Establishments. In some embodiments, the trusted relationship is for sharing pertinent information required for access to the Establishment, which may include health-related information. In some embodiments, the health-related information is provided in a one-time disclosure that is private and erased after a predetermined time following transmission. Designated Establishments have visibility and permission to communicate offers, incentives or other enticements to User(s) based on personal risk thresholds and schedule expectations. Basic data, on devices detected will be maintained and used for simple occupancy and traffic analysis at each Establishment; but no specifics of the User will be stored or maintained unless authorized by the User.

According to some aspects, the AQRM enables various safety measures to be enacted via alerts or controls reasonable to the Establishment (i.e. doors, gates, audio and visual alerts, etc.) to manage safety-related and social distancing measures for an Establishment.

According to one aspect, a method for providing real-time access, queue, and risk management information to a user is provided. The method includes receiving, from a user device, a request indicating a date and time a user intends to visit a first establishment. The method includes determining a predicted occupancy of the first establishment for the date and time indicated in the request using a predicted occupancy model (POM). The method includes determining an exposure risk for the first establishment for the date and time indicated in the request using an exposure risk model (ERM). The method includes transmitting, to the user device, the predicted occupancy and the exposure risk for the first establishment. The method includes receiving, from the user device, a confirmation that the user will visit the first establishment at the date and time.

According to another aspect, a method for providing real-time access, queue, and risk management information to an establishment is provided. The method includes obtaining in real-time or near real-time, from a data collection node located at the establishment, a data feed comprising one or more detected personal electronic identifiers (PEIs), wherein each of the one or more detected PEIs is associated with a monitoring device and the monitoring device is associated with an access level. The method includes determining an occupancy traffic at the establishment based on the detected PEIs. The method includes calculating occupancy parameters for the establishment defined by one or more third-parties. The method includes comparing the occupancy traffic to the occupancy parameters with respect to an establishment profile using a safety controls model (SCM). The method includes, in response to the comparing, transmitting an instruction to the data collection node to issue a command to an accessory device.

According to another aspect, an Access, Queue, and Risk Management (AQRM) system including a processor and a non-transitory computer-readable medium coupled to the processor is provided, wherein the processor is configured to perform the methods described above.

According to yet another aspect, a method for providing real-time access, queue, and risk management to an establishment is provided. The method includes obtaining, from a monitoring device, a message comprising a detected personal electronic identifier (PEI), wherein the monitoring device is associated with an access level and the PEI is associated with a device located in or near the establishment. The method includes transmitting, to a remote system, the detected PEI. The method includes obtaining an instruction to issue a command to an accessory device. The method includes issuing the command to the accessory device. According to another aspect, a communications, computing, command node, is provided. The node includes a processor and a non-transitory computer-readable medium coupled to the processor, wherein the processor is configured to perform the method.

### BRIEF DESCRIPTION OF THE DRAWINGS

FIG. 1 shows an overview of an AQRM system, according to some embodiments.

FIG. 2 shows a network overview of an AQRM system, according to some embodiments.

FIGS. 3A-3B show an example of an app screen output, according to some embodiments.

FIG. 4 shows an example database query, according to some embodiments.

FIG. 5 shows listed objects in a database, according to some embodiments.

FIG. 6 shows relationships between various data tables within a database, according to some embodiments.

FIG. 7 shows an example graphical user interface flow, according to some embodiments.

FIGS. 8A-8C show an example log-in screen, according to some embodiments.

FIGS. 9A-9B show an example safety rating screen, according to some embodiments.

FIG. 10 shows an example favorites allocations screen, according to some embodiments.

FIG. 11 shows an example risk threshold/risk budget screen, according to some embodiments.

FIG. 12 shows an example map screen of users, according to some embodiments.

FIG. 13 shows an example risk comparison screen, according to some embodiments.

FIG. 14 is a diagram of a CCCN/sensor module layout, according to some embodiments.

FIG. 15 is a diagram of a CCCN/sensor module layout, according to some embodiments.

FIG. 16 is a diagram of a CCCN/sensor module layout, according to some embodiments.

FIG. 17 is a diagram of a CCCN/sensor module layout, according to some embodiments.

FIG. 18 is a diagram of a CCCN/sensor module layout, according to some embodiments.

FIG. 19 is an architecture diagram, according to some embodiments.

FIG. 20 is a method for providing real-time access, queue, and risk management information, according to some embodiments.

FIG. 21 is a method for providing real-time access, queue, and risk management information, according to some embodiments.

FIG. 22 is a method for providing real-time access, queue, and risk management information, according to some embodiments.

### DETAILED DESCRIPTION

The AQRM system provides a number of technical advantages, including, inter alia:
(a) User control. The decision on whether to visit an Establishment is based on real-time data delivered to the User who can make decision(s) to visit based on their personal preferences and risk thresholds. The User has the sole power to establish and maintain a trusted relationship with an Establishment. The enhanced level of trust results in better communication, less transactional friction and better deals for the User. Users have complete control on the fate of data and must grant permission. All data that is not permitted to reside at either the Users' side or the business side of the system transaction is erased immediately upon completion of the interaction, transaction, or action. Establishments retain minimal information for pattern-based data analytics only. It is limited to that fact that an engagement occurred.
(b) AQRM is not a surveillance system. It is a sensing and data management system that delivers timely, relevant and actionable information to both Users and Establishments to save time and resources and to improve the overall experience. AQRM quantifies and provides visibility on occupancy activity and predictions on exposure risk, enabling Users to make informed decisions. The AQRM system enables Establishments to manage safety using pre-set limits based upon dynamic local and national safety guidelines/requirements disbursed via WiFi, BT, or other telecommunication methods and through the engagement of locally available alerts or mechanical safety devices.
(c) AQRM manages data in both a monitoring device and the Computing/Communications/Command Node (CCCN) with a balance between the two to manage latency. Significant computing and data management capability may be contained in the device electronics (chip sets, onboard memory and processing) to achieve low latency/lag in data transmission from Establishments to Users and vice versa.

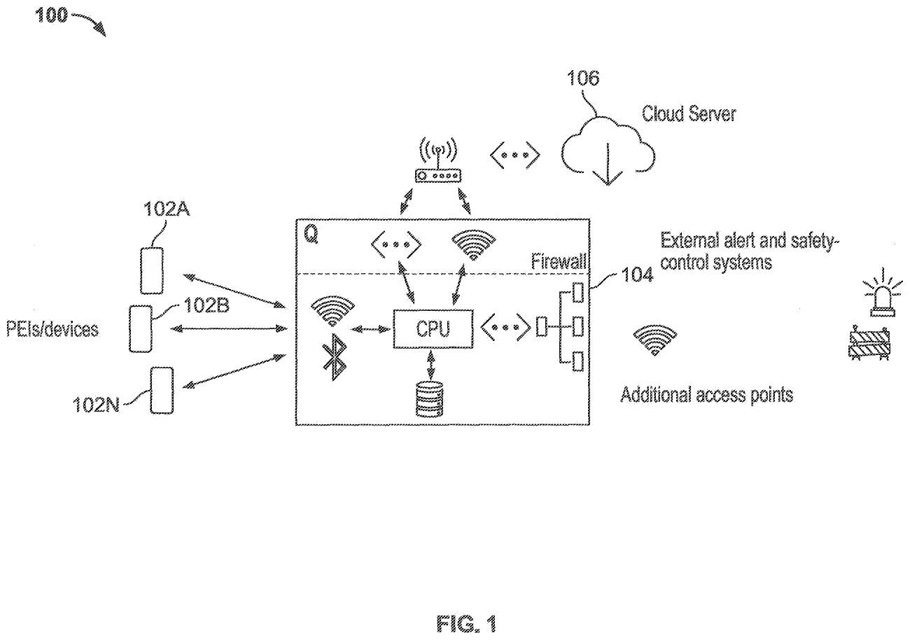

FIG. 1 shows an overview of an AQRM system 100, according to some embodiments. The AQRM system 100 may include a monitoring device 102A-N defined as, a two way radio device with antennae for directional/distance sensing, sound, video, utltrasonic or thermal sensing, data collection and relay capability using technology such as WiFi, BT, cellular, etc. required to collect and deliver data. The AQRM system 100 may include an on-premises Computing/Communications/Command Node (CCCN) 104 defined as the central electronics unit installed at an Establishment as the data collection hub for local data analysis purposes. In some embodiments, the CCCN may relay information to cloud-based computational analysis system 106, along with cloud servers, database software, artificial intelligence, and a mobile/web APP. The entire system enables the detection of PEI's, the analysis of data inputs and the reporting of both occupancy levels and exposure risk at each participating Establishment.

The AQRM system may include three main elements: (1) hardware, (2) cloud servers and software, and (3) mobile and web app, described in turn below.

Hardware

Hardware may be located on-premises at each participating Establishment and include a network of one or more BT/Wi-Fi (or other radio technology as developed) enabled monitoring device platforms (radio, optical cameras, sound, thermal imaging, ultrasonic sensors, etc.) placed at every ingress/egress point and at other interior areas to be monitored. Each monitoring device is associated with a physical location, such as Entry door 1, or an area, such as Break Room, and may be further assigned to an access level. All monitoring devices on the same access level comprise an access ring. Establishments may have multiple access rings. All perimeter monitoring devices are assigned to a level (e.g. 100, and others in interior space(s) are assigned to higher level access rings). Each monitoring device has the capability to detect and/or track multiple unique ID's from PEIs at a sample rate of dependent upon the needs of the system for data accuracy and system capability and communicates those detections to the on-prem Communications/Computing/Command Node (CCCN) which may also serve as the Wi-Fi portal to the cloud. Some embodiments may utilize monitoring devices that perform the same basic function as the CCCN and transmit data directly to the cloud. In some embodiments, such monitoring devices may be simpler versions of the CCCN. In some embodiments, the MQTT protocol may be used for transmitting data from monitoring devices to the cloud.

The on-board processor on the CCCN aggregates and synthesizes the data streams from all monitoring devices, maintains a contemporary database and identifies both the first and the last time that a PEI was seen by any monitoring device on a particular access level, pairing those times and locations to the PEI. The computing and processing capabilities on both the monitoring device and CCCN reduce the packet size, reduce latency to the cloud and provide immediate information to Users when necessary for shorter-term alerts and guidance. The onboard computing power of the CCCN or monitoring device may also provide basic computing and sorting of the data using algorithms that may allow direct access to information onsite prior to being further analyzed in the cloud.

The CCCN also receives instructions from the cloud and can issue commands to accessory devices that may control physical entry doors, to limit entry to those who have been granted permission by the system. This feature provides autonomous gate keeping and removes any human labor from the task of door monitor. The CCCN also sends pertinent information to any enabled Signage, or visual or audible devices “storefront display” within or outside the Establishment. The information might advise patrons (who are not Users) about occupancy status or wait times and provide details for them to join and become Users. In times of capacity constraints, entry preference goes to active Users since they are visible in the queue and have the appropriate credentials to pass through the entry gate.

Firmware/software systems may be programmed into the CCCN devices, e.g., sensors, to provide additional functionality. The firmware/software may include channel selection as needed during the site survey outcome, which allows the system to automatically select appropriate channels or be manually set at the site survey to allow for appropriate device detection thresholds. Utilization of one channel after an onsite survey determines the best channel for the site. The firmware/software can provide device clock synchronization with other sensing devices to allow for common data stream synchronization. The firmware/software enables migration from promiscuous mode to a more focused data acquisition mode when the data from the device is identified to a certainty that allows for accurate association of the device to a person or persons. There is a sniff mode versus message sending/definite identification and data sending, which allows for use of one antenna. Alternatively, the system can use two antennas and run promiscuous mode at the same time as the sending/definite mode

Cloud Servers and Software

Servers and software driven by algorithms manage the large amount of data collected by the monitoring devices, analyze it along with other inputs, to develop the prediction models and report occupancy levels and exposure risks to Users and Establishments alike, not only for the present moment, but for the foreseeable future (6-10 hrs. as an initial estimate with the potential for longer durations based on technological advancement).

Permanent data storage and high-level computing functions not performed within a monitoring device or centralized module (or CCCN), are managed in the cloud. Servers aggregate and synthesize detection data from all participating Establishments in conjunction with external data feeds to develop a clear picture of current conditions at each Establishment. Examples of external data feeds include: 1) prevalence of a communicable disease, such as Covid-19, by county and state mined from public health data sets, 2) data on occupancy restrictions from the latest public health advisories gathered by county and state, and 3) population data by county as well as other data sets.

The algorithm(s) perform four important tasks.
1) Predicted Occupancy Model (POM)—predict the near-term and foreseeable future (i.e. 6-10 hrs) at each Establishment based on User reservations and appointments in conjunction with current ad-hoc traffic and analysis of past occupancy trends. The prediction model is key to managing access and occupancy at each Establishment, to assess exposure risk, and to provide insight on anticipated demand to assist each participating Establishment with resource planning.
2) Exposure Risk Model (ERM)—develops and predicts an exposure risk metric for each User based upon their anticipated duration of interaction, the prevalence of a communicable disease, such as Coronavirus, in the community, the type of Establishment being visited and the predicted occupancy levels (from the POM) at the Establishment throughout their visit, along with other factors.
3) Cumulative Potential Exposure Risk Model (CPERM)—as Users interact multiple times with one or more Establishments, over a certain period, they accumulate an overall exposure risk. CPERM aggregates actual data from User interactions with the Establishments they visit to develop a cumulative exposure profile based on a rolling 14-day window.
4) Safety Controls Model (SCM)—is anticipated and claimed in this invention disclosure. It is an algorithm which utilizes a decision tree based on thresholds to engage existing or future installations of safety-related control measures (audible alerts, visual alerts, haptic alerts, gates, automatic doors, entry controls, guides, etc.) to manage ingress whenever current risk conditions exceed levels set by the Establishment or a User's personal preferences, etc. The public's movement in and out of Establishments is dynamic. The system continually monitors conditions at each participating Establishment and the evolving requests, defined as, an inquiry into the occupancy status of an Establishment. It is the first step in making either a reservation or an appointment by the collective User community. Artificial Intelligence (AI) functionality is employed to improve the quality and accuracy of model predictions. The updated information is distributed to both Users and Establishments through the mobile and web APP.

Algorithms, Data flow and function of AQRM system Predicted Occupancy Model (POM)

TABLE 1
Factors considered in Algorithm development and functionality

| Factors | Why?/Discussion | Proxy | Data Source |
|---|---|---|---|
| Historical | Establishments tend to | Historical records | QF-Detect data for comparable |
| occupancy traffic | have repeatable, | of devices | period (DOW, TOD, etc.) |
| (for comparable | explainable, and | (phones, PEI) | |
| period) | predictable patron | detected in the | |
| DOW/TOD/Month | traffic. | Establishment for | |
| to reflect seasonality | | comparable | |
| | | period | |
| Standard Dev. of | The accuracy of | STD Dev of each | QF-Detect data for comparable |
| Historical activity | prediction is driven by | 15 min. segment | period (DOW, TOD, etc.) |
| for the period | the dependency of the | of relevant | |
| selected. | historical record. | occupancy data | |
| | Tightly clustered data | on the | |
| | provides greater | Establishment | |
| | accuracy | | |
| Current detection | Current activity taking | Current activity | QF-Detect data for |
| activity occurring at | place 1 hr. prior to | detected at | Establishment for period of T-1 |
| Establishment | query is used for | Establishment | hr. before request |
| | comparison against | | |
| | historical to project | | |
| | trend to establish | | |
| | multiple Time- | | |
| | dependent scaling | | |
| | factors to develop the | | |
| | POM | | |
| User's Anticipated | POM works to | Request Time | QF-Requests table |
| time of interaction | determine an occupancy | Stamp, Request | |
| | trend for the time period | Entry Date/Time | |
| | selected by the User. | for interaction; | |
| | | Duration(Estimated | |
| | | length of stay) | |

Initial Algorithm-Predicted Occupancy Model (POM)
1. Use inputs from Requests to establish parameters for data retrieval of any records from the historical detect data from the Establishment that are comparable in terms of DOW, Month, etc. Output two arrays: A) Request Time Stamp to T−2 Hrs (currently at 15 min. intervals, B) Request Entry Date/Time to (Request Entry Date/Time+Duration).

Calculate Std Dev. on both arrays, for each 15 min segment. Average the Std. Dev over array length. Value used in steps 4, 6 and 8 to evaluate sample accuracy.
2. Determine correlation and scaling factors of array A against detection activity for the current period bounded by Request Time Stamp to T−2 Hrs. (9 sample points @15 min. intervals.).
3. TEST: If correlation >0.70 or STD DEV<2, then pass small sample and go to step 9.
4. In the absence of viable historical trend (array A), retrieve and test data from this Establishment for an aggregate period (increase selection parameters to previous xx days). Repeat step 3.
5. TEST: If correlation >0.70 or STD DEV<2, then pass Establishment aggregate sample and go to step 9.
6. In the absence of viable aggregate trend from this Establishment, retrieve and test from similar Establishments in the QF population for an aggregate period of xx days. Repeat Step 3.
7. TEST: If correlation >0.70 or STD DEV<2, then pass similar Establishment aggregate sample and go to step 9. Otherwise state no comparative data available.
8. Use these tests (steps 4, 6 or 8) to determine which sampling set will be used to pull Array B. Determine Std. Deviation in final array B. This value will be used to judge confidence.
9. Calculate array C for the corresponding interaction period from Array B using scaling factor produced from associated Step 3. Output Array C for use in ER Model.

The Std. Deviation of the selected Array B and Correlation value from the final selected sample of Array A provide the degree of confidence in the validity of Array C.

Array C provides predicted occupancy levels to ERM that match the interaction time selected by the User.

PO-Array C=From: Request Entry Date/Time To: (Request Entry Date/Time+Duration): PO=Roundup(Array B*Z,0)

Z—scaling coefficient derived from comparisons performed in Step 3.

Machine learning and analysis will be used to refine acceptable limits for correlation and Std. Dev.

Exposure Risk Model

TABLE 2
Factors considered in Algorithm development and functionality:

| | | Proxy | |
|---|---|---|---|
| Factors | Why?/Discussion | Variable(s) | Data Source |
| % of Pop infected | Prevalence of disease, | Positivity rate/ | EXT-CDC, States and other |
| | but difficult number to | test conducted | provide external feeds on testing |
| | know. Because | Positives/100K | |
| | infection data unknown | Pop | |
| | | 7d M avg. for | |
| | | both | |
| Dwell time | Length of exposure risk | Duration | QF-Detected and predicted |
| | | | engagement data (POM) |
| | | | (calculated in 15 min segments |
| | | | to factor in changes in |
| | | | occupancy affecting density and |
| | | | distancing |
| Density of people | Probability of | Projected | QF-profile data on |
| | encounters. Usually | Occupancy/ | Establishments |
| | expressed as X sq. ft./ | Posted | |
| | person. Difficult to | Occupancy | |
| | know easily. Using | | |
| | posted OCC per fire | | |
| | code follows established | | |
| | guidelines from NFPA. | | |
| Distancing expected | See 3) Too hard to | Projected | QF-profile data on |
| | know values for each | Occupancy/ | Establishments |
| | Establishment. Default | Posted | |
| | to values developed by | Occupancy | |
| | NFPA | | |
| Airflow (ventilation) | Air exchange plays an | Establishment | QF-profile data on |
| | important role in how | Type | Establishments and |
| | long viral particles | | specification of type according |
| | linger in air. Specific | | to definition |
| | data will be too hard to | | |
| | gather on per location. | | |
| | Use assumptions based | | |
| | upon Establishment | | |
| | type | | |
| Type of activity | What people are doing | Establishment | QF-profile data on |
| | in a space matters to | Type | Establishments and |
| | their propensity to shed | | specification of Estab. type |
| | viral load, if | | according to definition |
| | infected. Best estimate | | |
| | is based upon average | | |
| | assumed activities in a | | |
| | type of Establishment | | |
| Personal mask | Mask use by the User is | Establishment | Q-profile data on |
| effectiveness | often driven by type of | Type | Establishments and |
| | activity and the type of | | specification of type according |
| | place | | to definition |
| Mask efficiency/use | Mask use by others is | Establishment | Q-profile data on |
| of others | often driven by type of | Type | Establishments and |
| | activity and the type of | | specification of type according |
| | place they are at. For | | to definition |
| | instance, restaurant and | | |
| | bars = low use, grocery | | |
| | store and big box tend | | |
| | to higher use. | | |
| | Too hard to quantify | | |
| | assume by | | |
| | Establishment type | | |

Initial Algorithm—Exposure Risk Model (ERM)

ER score is required to provide individual Users information regarding their personal risk based on their preferences.

ER=A*(Total tests)*(7d_MA_Positivity)\hat{ }B+C*(7d_MTot_Prevelance)\hat{ }13/(1/Gathering Limit)+E*((Projected or actual OCC)/Posted OCC)\hat{ }F+G*(Estab. Type Risk Factor).
1. 7d_MA_Positivity—7 day moving average of positive test results/total tests performed.
2. 7d_MTot_Prevelance—7 day moving total of positive cases/100K of population.
3. A, C, E and G—scaling coefficients to address relative importance/contribution of each factor.
4. B, D and F—power factors to address the geometric increase in chance/impacts from either more people being infected or others being closer to you. (area is defined as sq. feet, so it makes sense to power the derivative terms of occupancy.
5. Machine learning and analysis will be used to adjust scaling coefficients and power factors.
6. The ebb and flow of people is variable and volatile, making it difficult and inaccurate to project the ERM to extended periods of time. Accordingly, time was segmented into 15 min. increments to calculate ER in discrete segments using time-specific variables.
7. Overall exposure risk (ER) is a sum of these discrete time segments either experienced, or projected, from an interaction with an Establishment.

Cumulative Potential Exposure Risk Model (CPERM)

TABLE 3
Factors considered in Algorithm development and functionality

| | | Proxy | |
|---|---|---|---|
| Factors | Why?/Discussion | Variable(s) | Data Source |
| Actual | Exposure is a function | Actual | QF-Detect data |
| occupancy | of interaction, density, | occupancy | |
| traffic (for | and time. | detected at | |
| comparable | | Establishment | |
| period | | | |
| of Users | | | |
| Interaction) | | | |
| User's length | Exposure is | Duration of | QF-Detect |
| of Interaction | a functional | interaction | data filtered to |
| | actual time spent | | User's request |
| | interacting | | |
| Risk factors | Exposure risk is a | ERM model run | QF-Detect |
| calculated | function of prevalence | using actual | data for |
| and used | of disease, density and | duration and | Establishment |
| in ERM. | Establishment types on | occupancy | ERM-see |
| | whether people | data at | A.1.2. |
| | maintain distance and | Establishment | |
| | mask use. Thse factors | | |
| | were utilized in | | |
| | developing the ERM. | | |
| Accumulated | Users make multiple | | |
| Exposure | visits to multiple | | |
| | Establishments over a | | |
| | 14-day period | | |
| | (considered the current | | |
| | incubation window for | | |
| | COVID). Their risk in | | |
| | not just a function of 1 | | |
| | interaction, but of | | |
| | many during a rolling | | |
| | 14-day period. | | |

Initial Algorithm—Cumulative Potential Exposure Risk Model (CPERM)
1. Use calculated values from ERM for the periods of engagement by User(s) Request Entry Date/Time to (Request Entry Date/Time+Duration) ACTUAL Values.
2. Retrieve actual detected data for occupancy at the Establishment(s) for corresponding time frame.
3. Retrieve actual duration of User interaction from detect data.
4. Calculate actual exposure risk for this interaction and log on request table.
5. Repeat process for all other interactions
6. Filter and sum all User ER calcs on rolling 14-day period and maintain on User profile. Calculate daily and store values on User history table.

Safety Controls Model (SCM)

TABLE 4
Factors considered in Algorithm development and functionality

| | | Proxy | |
|---|---|---|---|
| Factors | Why?/Discussion | Variable(s) | Data Source |
| Actual | Exposure is a function | Actual | QF-Detect data |
| occupancy | of interaction, density, | occupancy | |
| traffic (for | and time. | detected at | |
| comparable | | Establish- | |
| period | | ment | |
| of Users | | | |
| Interaction) | | | |
| Risk factors | Exposure risk is a | ERM model | QF-Detect |
| calculated | function of prevalence | run using | data for |
| and used | of disease, density and | actual | Establishment |
| in ERM. | Establishment types | duration and | ERM-see A.1.2. |
| | on whether people | occupancy | |
| | maintain distance and | data at | |
| | mask use. | Establish- | |
| | These factors | ment | |
| | were utilized in | | |
| | developing the ERM. | | |
| Occupancy | Occupancy | | QF-Establish- |
| parameters | restrictions may | | ment parameters |
| defined for | require Establish- | | QF-ERM |
| Establishment. | ments to exert | | QF-Govt Rest |
| Government | control over entry in | | QF-Detect Data |
| requirements | order to maintain | | |
| or guidelines | social distancing. | | |

Initial Algorithm—Safety Controls Model (SCM)
1. Retrieve real-time detected data for occupancy at the Establishment(s).
2. Calculate current social distancing and occupancy restrictions upon Govt restrictions/guidelines using data feeds.
3. Compare to real-time occupancy and parameters from Establishment profile.
4. Send alerts to Establishment and Users.
5. Send commands to crowd control devices (auto entry doors, etc.) and actively begin to manage ingress to Establishment.
6. Re-direct Users who wish to engage to AQRM queuing functionality to reserve a place in queue.
7. Monitor changes to occupancy and admit Users when levels fall below pre-determined values.

Cloud resources aggregate and manage the multitude of requests for reservations or appointments by every User for all participating Establishments on the platform. The system manages User(s) request(s) based on their personal risk thresholds and preferences and can develop a working schedule to plan out a User's day of multiple interactions with the goal of minimizing exposure risk, or avoiding a wait in a queue. Other existing platforms, databases, and/or hardware may also integrate into AQRM resources.

The software systems may be located on a cloud server, individual computers, or other distributed computing systems 106. In some embodiments, the system may use a MQTT, or similar, messaging protocol to manage the signals from the devices to the central data processing server/center and/or as the central data processing server/center. The system may utilize a bi-directional communication and provisioning protocol to manage the data flow from CCCN/sensors to the computing platform, such as, for example, MQTT.

In some embodiments the system 100 monitors the environment for WiFi management frames. RSSI may be used to establish a local region of interest within a given establishment in which devices will be monitored for presence.

In some embodiments, the system 100 establishes overlapping local regions to allow for the differentiation among multiple devices per person. Counting MAC or other device addresses alone may overcount the individual occupancy, and one advantage to establishing local regions is to allow for the allocation/counting of devices versus users both spatially and temporally.

In some embodiments, the system maintains a database which contains known devices (i.e. local printers, TVs, or registered users who are trusted by the system) and discovered devices (i.e. new devices to the system related to potential new users and new occupants). The system updates the presence of an identifying address of any device when a new device is detected. In some embodiments, the system has functionality to ping new addresses related to new devices to trigger a potential trusted engagement with the new potential user. A processing event may be triggered when a new device is detected with the goal of generating a message to server that only contains likely devices associated with people to minimize over estimation of people versus devices.

In some embodiments, the software system utilizes algorithms and rules to sort devices versus MAC addresses or other identifiers to provide a high level of accuracy of association between occupants and devices to minimize the data processed in the database to generate the onsite occupancy, risk, etc. analysis.

The sensing devices are configured in a variety of ways for set-up at an establishment and/or field deployment. Status messages may be to the server to alert for potential maintenance issues.

In some embodiments, Users may be known to the system by means of an opt-in. In some embodiments, one or more software algorithms may be used to detect individuals. For example, two or more signal formats may be used to detect individuals. Device identifiers may be accumulated based on time to estimate an individual with acceptable certainty, even if signals and associated identifiers are constantly randomized per ping. Individuals may be identified based on definite device identifier certainty with either a definite ingress/egress time stamp or a time-out function that assumes the individual has left the premises if not identified again. Identification of individual devices may be acquired based on other “wireless” markers such as wireless charging identification/authentication processes, device-specific signal characteristics, human usage patterns, etc. In addition, a digital ID discriminator may be used to mark an individual device, relate that device to a person or persons, but not track or store information particular to that device which is initiated by the user as a trusted method for allowing local marking and identification to be passed in a single usage format to the establishment.

Mobile and Web APP

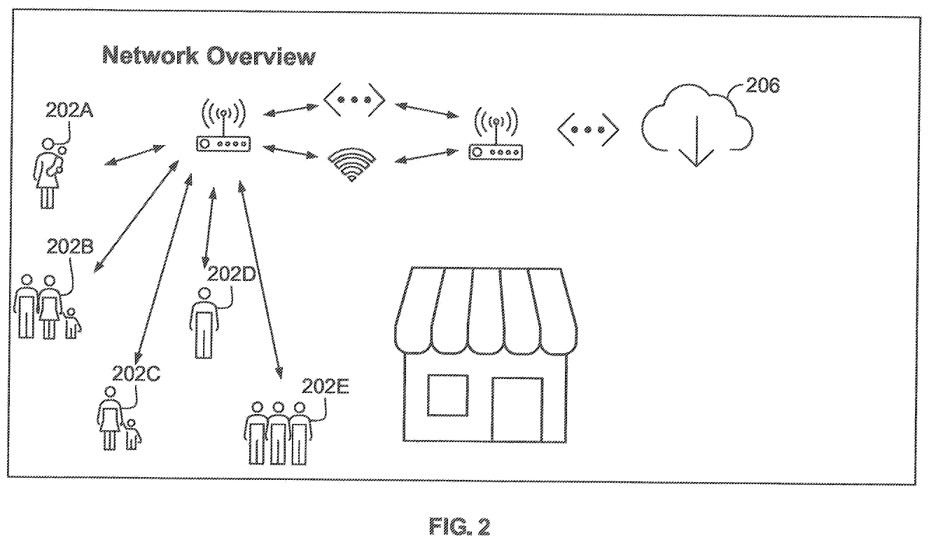

A mobile/web-based APP is the link between the User(s), the Establishment(s) and the cloud resources. FIG. 2 shows a network overview of an AQRM system, according to some embodiments. Users 202A-E build a profile making preferences, setting personal risk thresholds, and selecting which Establishments are “trusted”. Once their profile is created, Users actively search and select participating Establishments and the dates and times for their possible interactions. These details upload to cloud resources 206 and initiate queries with the predictive models. The resulting output on occupancy levels and exposure risk is seamlessly downloaded and displayed via the APP to assist the User in making informed, risk-based decisions while planning interactions. Since the system is cloud based, Users have access to this information far away from the Establishment.

Establishments also use the APP to build a profile for their location inputting information on the type of Establishment, posted occupancy (which may be defined as, Maximum posted occupancy allowed at each Establishment determined by the guidelines of the National Fire Protection Association (NFPA) and local Fire authorities), the hours of operation, etc. This information, together with the detection data, is uploaded to the cloud and is used to understand current conditions and to predict occupancy for the foreseeable future. Establishments can monitor their occupancy flow, both present and planned, and can readily make informed decisions, if conditions change. They can decide whether to send notifications (appointment/reservation availability, acceptable risk level), etc., or offer incentives (i.e. a discount based on rewards) to Users that have enabled trusted relationships.

In some embodiments, an app or computer-based system includes risk management/budgeting features. The features may include a risk budgeting system designed to provide and manage an allotment of time or user (individual) involvement or location in various situations to reduce risk of exposure to infectious, transmissible diseases. Such a system may include the capability to allow for one or more of the following functions.

(User and Establishment) Time-based, occupancy-based components combined with local risk calculations and factors to create a risk assessment for users.

(User and Establishment) Management and reporting risks to users and establishments; offering alternatives to users based on other known activities or establishments that may better suit their risk expectations.

Permitting a user to set a “cumulative exposure budget and risk assessment” that provides alerts to the user regarding the usage of the budget throughout a planned period; alerting to a user to a change in the occupancy conditions of the establishment if, while a user is en route to the establishment, their risk parameters are exceeded with a corresponding offer for placing a take-out or delivery order instead.

In some embodiments, a system for establishments is provided to manage their risk information to the latest local conditions to re-set the lay-out of their establishments to address conditions in real time.

(Establishment) A system of reporting to local officials the data required to allow for establishments to remain open with remotely approved plans and reporting. Based on local occupancy standards/mandates, the system could be equipped to transmit data to regulatory bodies to show compliance with such standards/mandates. Having such a system could allow for an establishment to remain open and active without having to have an onsite inspection by a local official. As standards/mandates change, the establishment could modify and report their onsite setup and occupancy management “on the fly.” Such a system would allow the transmission of a map of the seating arrangement to both users and local officials for remote approval based on the occupancy required to meet the immediate safety codes and conditions.

In some embodiments, a system is provided for inputting vaccination/immunity data into the personal user's system to lower risks for the individual user on a personal, private, secure basis. Vaccination status versus recovered/not recovered from the virus interacts with local risk calculators (or default QFirst-developed) calculator to enhance the risk prediction and risk budget baseline. Different variants of a virus can be uploaded and assessed in the risk profile database as QFirst develops or taps into a database appropriate to modify the risk budget accordingly.

In some embodiments, a system is provided that allows a user to transmit securely vaccination/immunity information to an establishment on a per use, individual basis as an encrypted and targeted “ping” to an establishment allowing entrance or a different disposition determined by the user's health status. This system can influence reservation requests (priority), the purchase of tickets or other entrance passes, based upon the health profile of the user. The user can securely upload basic health data to system, and then opt to share simple YES/NO metrics for future use and reference by the establishment to offer enticement to visit the establishment given the user's known risk budget and threshold settings and the relative health profile. For example, a risk budget and risk threshold of a certain level can allow an establishment to make a reasonable inquiry to a user to come to the establishment for a visit based on the budget and risk threshold of the user along with an enticement of discount or other preferred service.

(Users and Establishments) In some embodiments, an app-based calculator is provided to assess chances of CoVID-19 exposure based on one or more of: exposure risk budget calculations for the immediate area; local area conditions; conditions of an area to which the user is traveling. The system may include tailorable alarms and alerts to allow user to make decisions concerning risk.

Functional Scenario—User

Users may download the APP from various sources (on-site QR codes, marketing materials, email campaigns, texted links, shares by friends or family, etc.), complete the User profile and select their preferences for risk thresholds from various options on the APP. These preferences help the AQRM system guide their decision-making process while making a reservation or an appointment.

A User can then plan a visit by opening a request, selecting a participating Establishment and specifying a desired date and time for a potential visit. The APP sends this info to the cloud and returns with a status of the current conditions at the Establishment and a forecast of both occupancy and exposure risk for the entire time of their proposed visit (interaction). The User reviews the info and makes any adjustments to fit their needs, schedule and/or risk tolerance. Their adjustments cause interactive updates, so the User sees the latest info. Once finalized, the User simply presses the COMMIT button on the APP to move the request to a reservation, or appointment. From that point on and through their departure at the Establishment, the User is provided with regular updates from on-going data analytics to help them adjust their decisions based on any changes may occurring at the selected Establishment.

The User builds their personal interaction itinerary by repeating this request/reservation process for any other Establishments they may want to visit. As they commit, they receive updates on each of the added Establishment(s).

Users may wish to make an appointment for a future date and time which may be out in front of the occupancy information and predictive models. In this scenario, the system will record their request/appointment and will show what is currently known about occupancy based on other appointments. As the appointment date and time gets to within the range of the models (i.e. 6-10 HRS), the User will automatically begin to receive updated information on their scheduled and pending interaction.

Users can also create “trusted” relationships with Establishment(s) of their choice. It is solely User-driven and enables User(s) to have greater communication with chosen Establishments they like or happen to visit frequently. They receive notifications and incentives that conform to their preferences and may also receive special alerts only available to trusted relationships.

### Functional Scenario—Establishment

An array of monitoring devices and the Communications/Computing/Command Node (CCCN) module are installed at the Establishment. Operators create an account, build a profile with information specific for their location, associate each monitoring device to its physical location (i.e. Entry door #2, Break room, etc.) and assign it to an access level during the set-up process.

The monitoring devices detect the presence of all PEI's in range within the Establishment and communicate that data to the CCCN which processes and sends information on current conditions to the cloud resources. If a detected PEI belongs to a registered User, they also receive an alert to notify them that the system has detected their presence and they will receive any necessary notifications or instructions. If a detected PEI, is a Non-registered ID (NRID), defined as, a device that has been detected and indicates the presence of at least one person in the physical form, but is not yet registered with the AQRM system, then the system will provide instructions to the NRID.

In instances where the system interfaces with auxiliary equipment, such as automatic door openers, door latches or display devices, the CCCN is paired to these devices using the APP so that any commands or communications can be sent seamlessly to these devices.

The APP provides a dashboard to Operators at participating Establishments enabling them to manage: 1) on-site occupancy expectations and capacity (based on changing occupancy guidelines), 2) information published about their Establishment which may assist Users when planning a visit, 3) ingress/egress counting and entry control autonomously, without human labor, and 4) notification on the arrival of Users with reservations/appointments.

Operators can also view comparisons of current activity vs. historical for the corresponding period and see the effect of various events or external factors such as weather vs. predicted customer traffic.

Establishments can also adjust their business practices, configurations, table lay-outs, etc. based on the analysis of User risk profiles. For example, trending data may indicate that people are most comfortable with tables of four and that requests for large tables of 6-8 are minimal. The Establishment can then optimize seating. They gain value in learning how to tailor their approach to manage risk given their unique situation and provide a safe environment for their customers.

Forecasting is another important tool provided through the APP. In the past, many Establishments relied solely on foot traffic without any reservation system or visibility to plan resources. This invention increases the visibility into future interactions by showing all scheduled appointments and reservations making actionable demand planning possible.

At times when an Establishment's capacity is exceeded, the system automatically routes Users to a virtual queue and keeps them updated and engaged during the wait. Users can continue with other activities and then interact the appropriate time with little friction. When a situation arises that exceeds pre-set risk levels of either the User or the Establishment, the system can activate various forms of pre-existing or future-installed safety controls(alerts, gates, guides, door operation, notices, etc.) to heighten awareness or enable the User and/or Establishment to control their risk.

Establishments benefit from having comfortable customers and those with the “trusted” designation by a User, gain additional insight and connection. Operators see a User's visiting behavior pattern along with their preferences. They can then send notifications or incentivize each User with information on immediate availability, a better time to come to avoid crowds or a sweeter deal. Using technology to flatten the demand curve and control occupancy and social distancing practices of the Establishment while reducing the transactional friction for a User improves the possibility of a visit, enhancing the User-to-Establishment relationship.

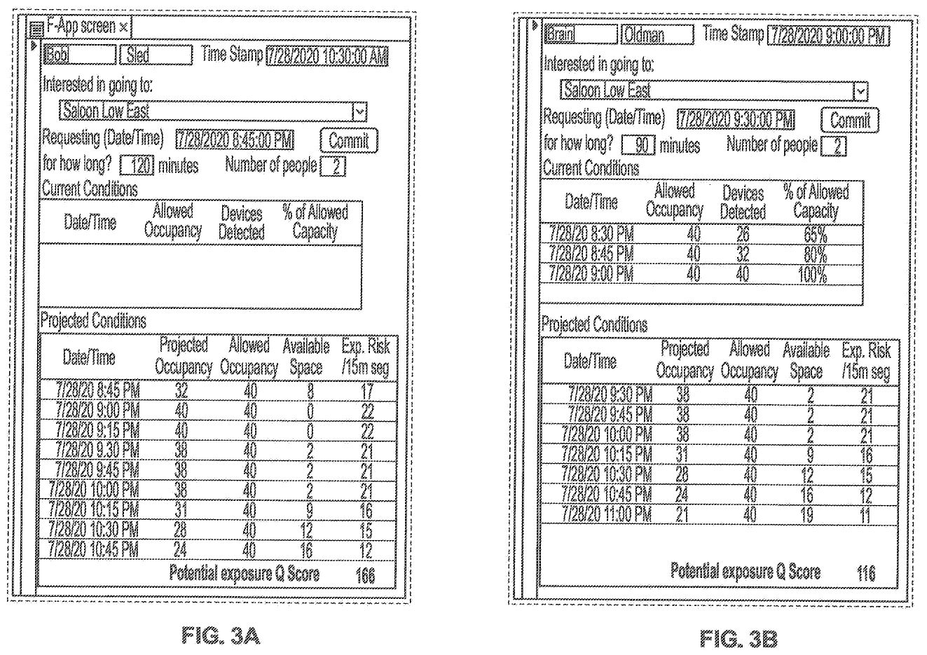

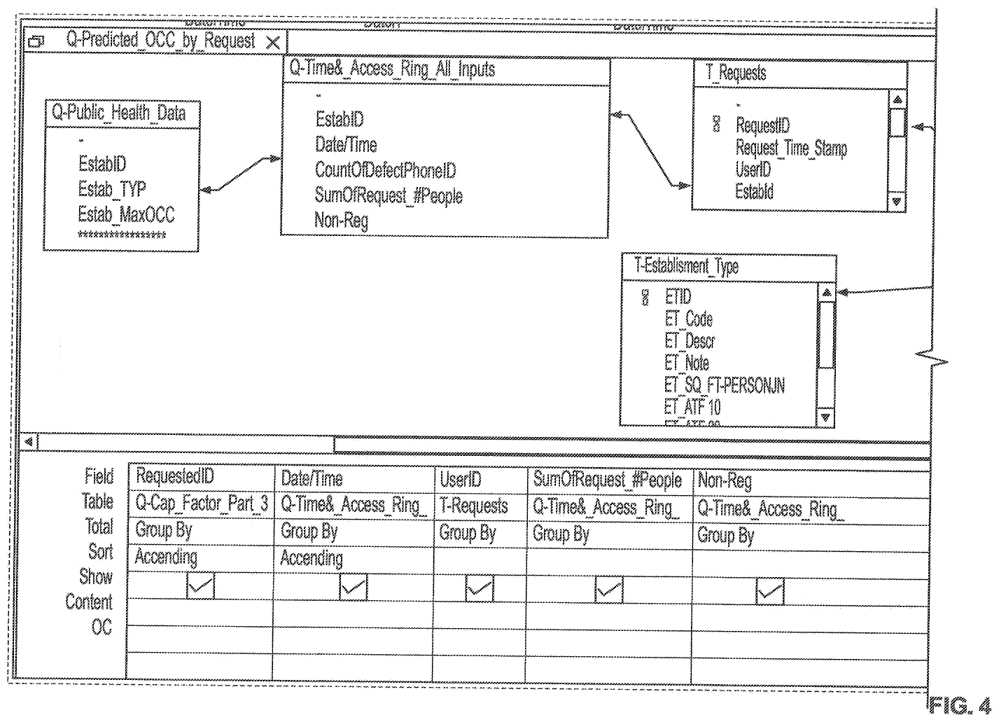

FIGS. 3A-3B show an example of an app screen output, according to some embodiments. FIGS. 3A-3B show projected conditions displayed for the anticipated duration for the User's visit. Difference in the ER score is driven by a function of User's duration of interaction and occupancy loading of Establishment. FIG. 3A shows an example screen output in response to a request made very early in the day, hence there was no activity to populate the current conditions which looks at the hour ahead of when you are making the request. FIG. 3B shows an example screen output in response to a request made within ½ hour of desired interaction. Current conditions are visible for the prior hour.

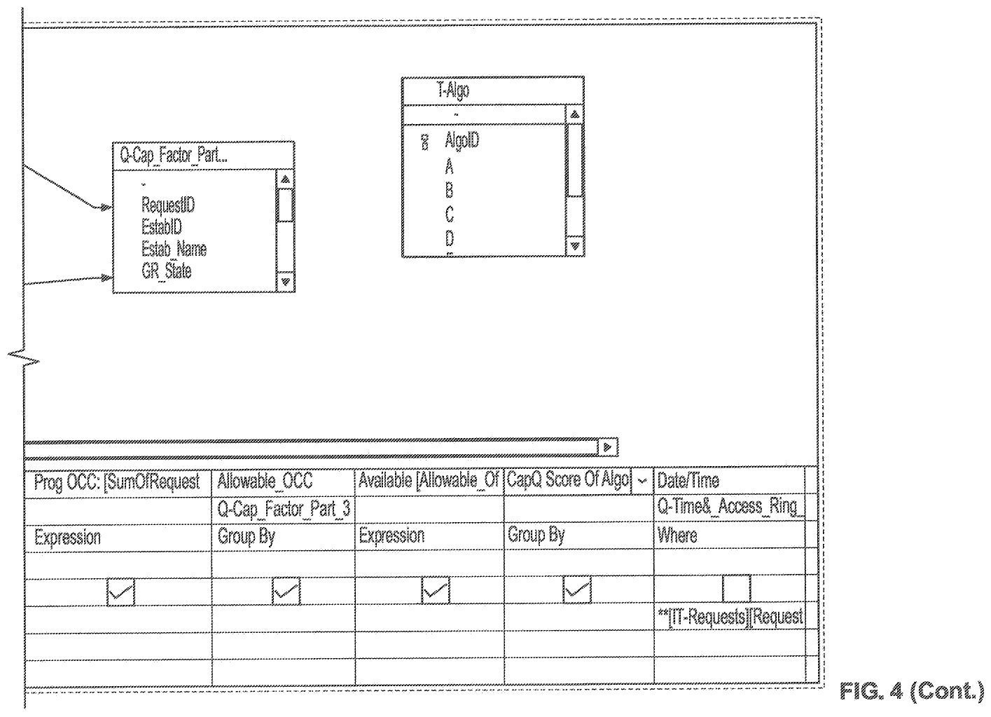

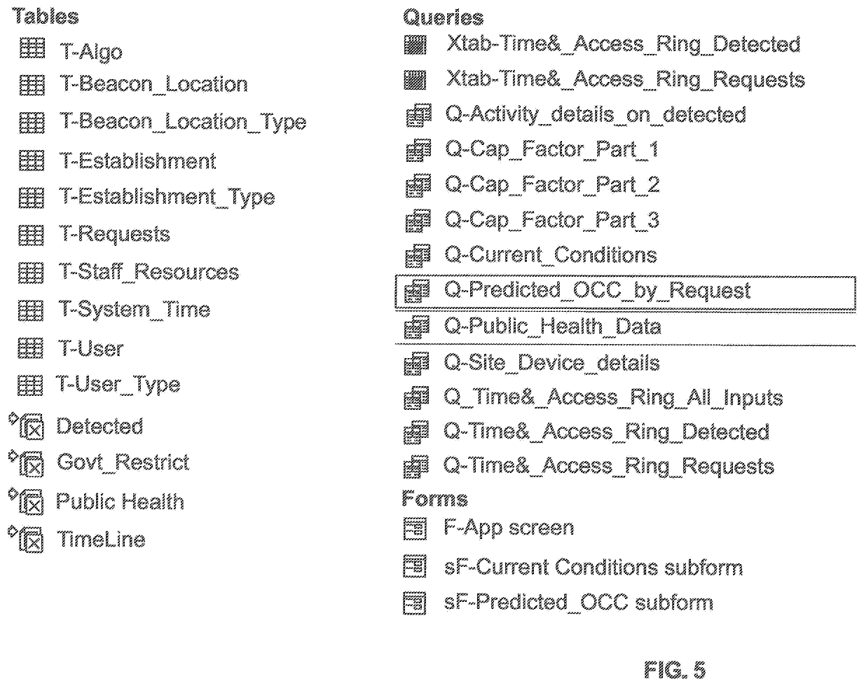

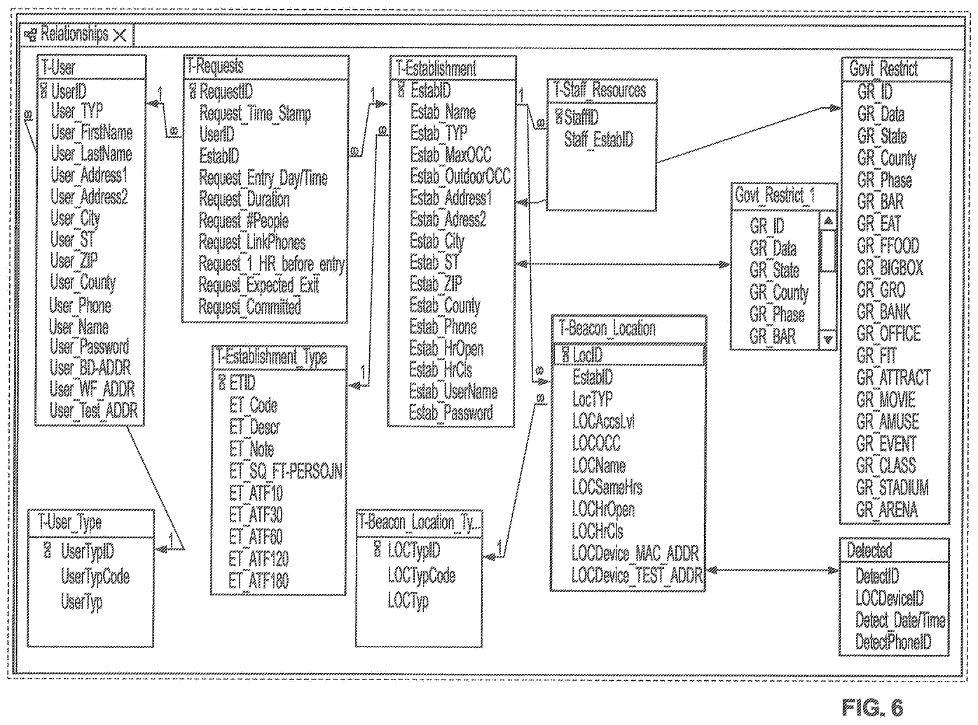

FIG. 4 shows an example database query, according to some embodiments. FIG. 5 shows listed objects in a database, according to some embodiments. FIG. 6 shows relationships between various data tables within a database, according to some embodiments. In some embodiments, the database in FIGS. 4-6 is an AQRM database. According to some embodiments, the objects in the AQRM database may include external data sources reflecting API integration, such as tables “Detected,” “Govt-Restrict,” “Public_Health,” and “TimeLine.”

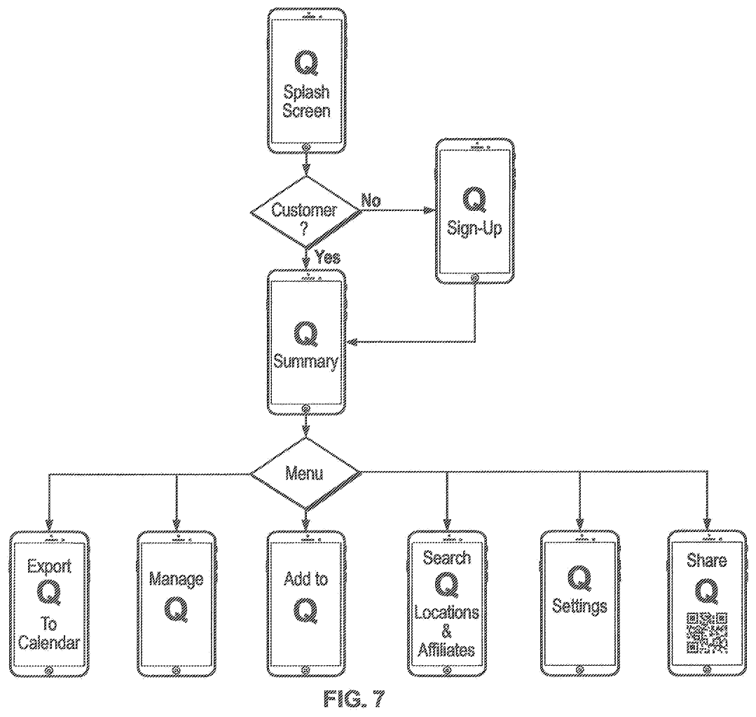

FIG. 7 shows an example graphical user interface flow, according to some embodiments. According to some embodiments, the GUI may be for the AQRM App, and may be supported on several platforms including, for example, iOS, Android, and Java.

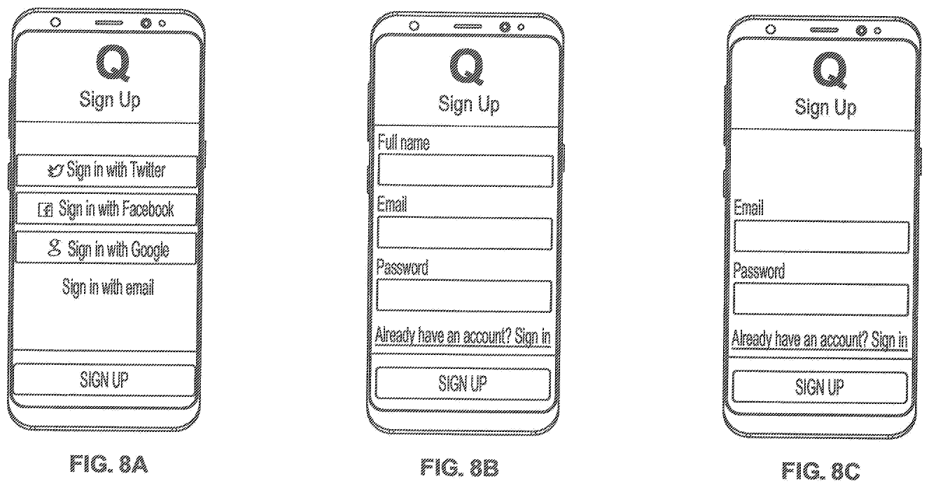

FIGS. 8A-8C show an example log-in screen, according to some embodiments.

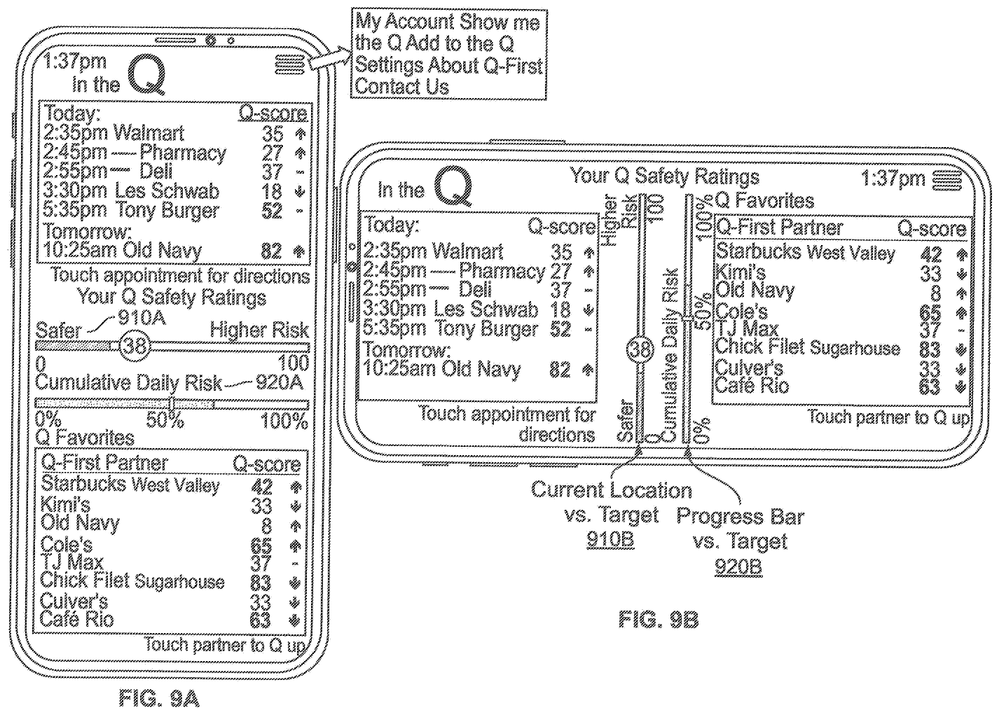

FIGS. 9A and 9B shows an example safety rating screen, according to some embodiments. FIGS. 9A and 9B illustrate a series of Establishments and Q-Scores associated with each Establishment. The safety rating screen may include an interactive user interface element, such as a slider bar, that indicates a safety rating for a current location v. target location (910A, 910B), and a cumulative daily risk (920A, 920B). The safety rating screen may further include a list of favorite establishments and a respective Q-score.

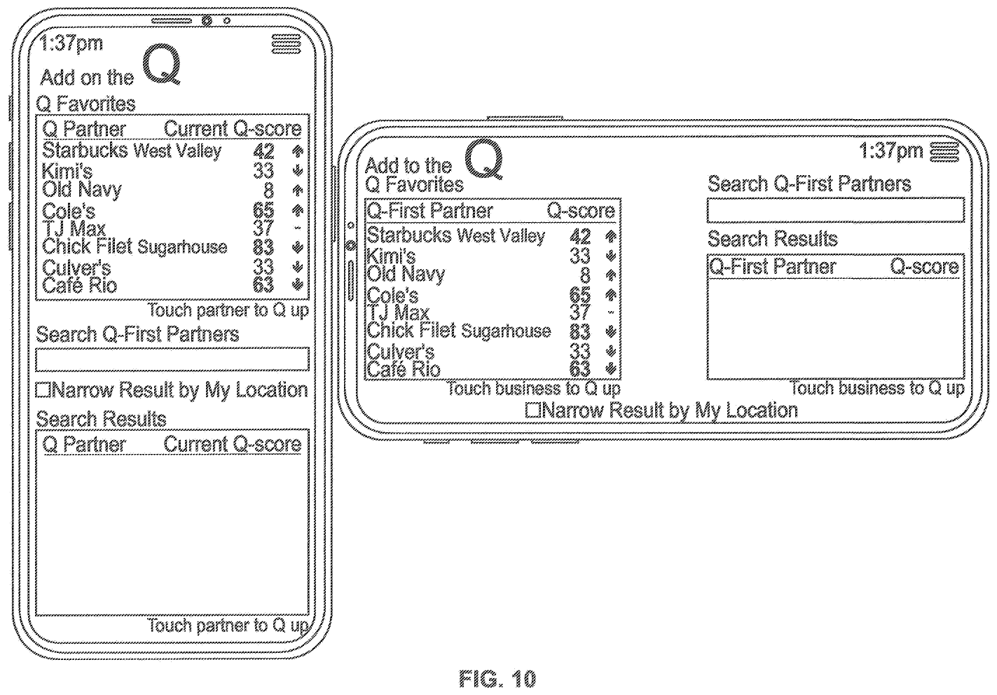

FIGS. 10A and 10B show an example favorites screen, according to some embodiments. FIG. 10A includes a favorites screen in portrait mode and FIG. 10B includes a favorites screen in landscape mode.

Additional GUI screens may include, for example, a map of Q-partners nearby, risk factor comparisons (e.g., “more likely to get hit by lightning,” “more likely to get eaten by a shark,” “more likely to get hit by a car walking,”) among others.

According to some embodiments, the skin of the GUI may be dynamic and customizable for each client. The Establishment in the database may contain, for example, an image of an Establishment's logo and parameters on a color palette, which will be used to change the GUI display as users select the Establishment. The GUI skin customization may reinforce a User's familiarity and connection to the Establishment.

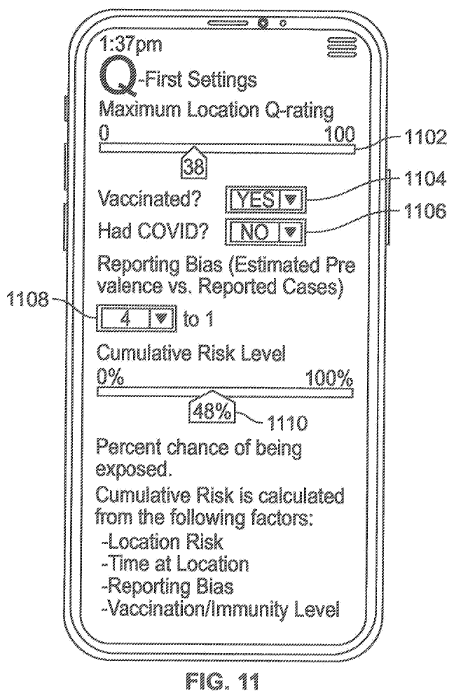

FIG. 11 shows an example risk threshold/risk budget screen, according to some embodiments. According to some embodiments, Users may be able to establish a risk/threshold risk budget in connection with using the App. According to some embodiments, a User may be able to indicate one or more of: a maximum location Q-rating 1102, e.g., on a scale of 0 to 100; whether the User has been vaccinated or not 1104; whether the User has had a communicable disease, such as CoVID 1106; a reporting bias 1108 (e.g., estimated prevalence of disease v. reported cases); and a cumulative risk level 1110, which may be based on one or more of location risk, time at location, reporting bias, and vaccination/immunity level.

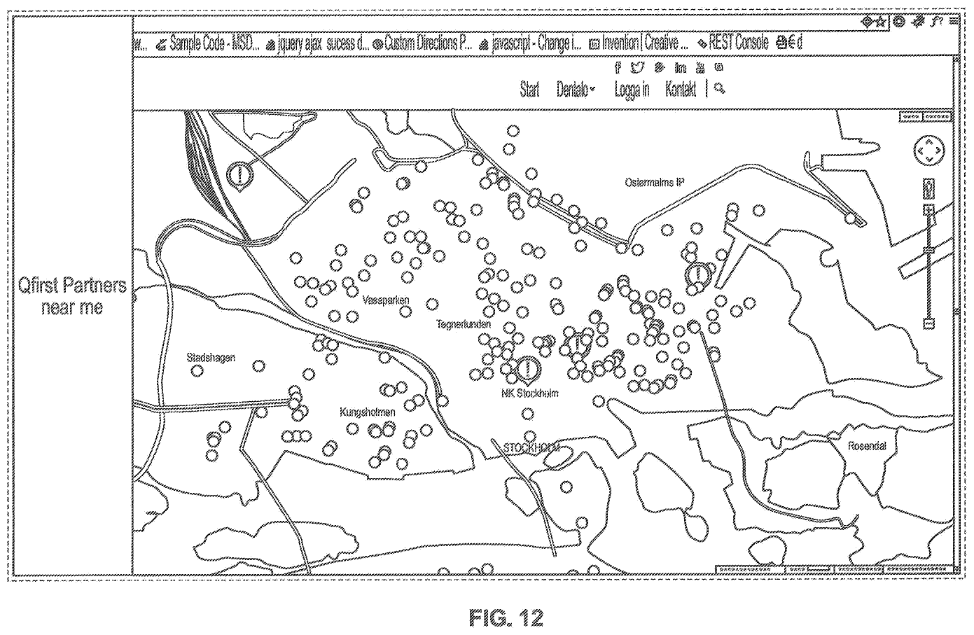

FIG. 12 shows an example map screen of users, according to some embodiments. According to some embodiments, the App may display a map of partners and participating Establishments nearby.

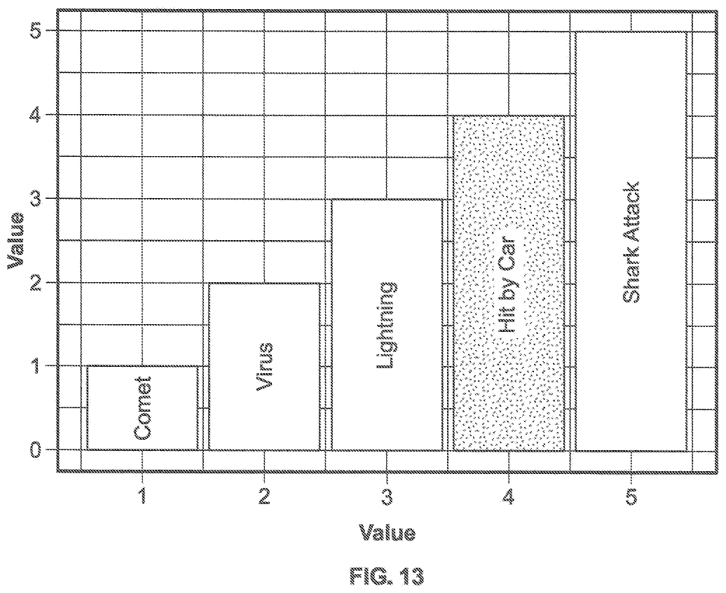

FIG. 13 shows an example risk comparison screen, according to some embodiments. In some embodiments, the app may rank a User's “viral” risk in comparison to other known risks in the area. Each risk may be highlighted in order to show local trends and information. Different colors could signal most common risks in the immediate area. FIG. 13 illustrates alternative risks including a comet, virus, lightning, hit by car, and shark attack, with “hit by car” risk in highlighted in a different color to signify that the risk is more common in the immediate area.

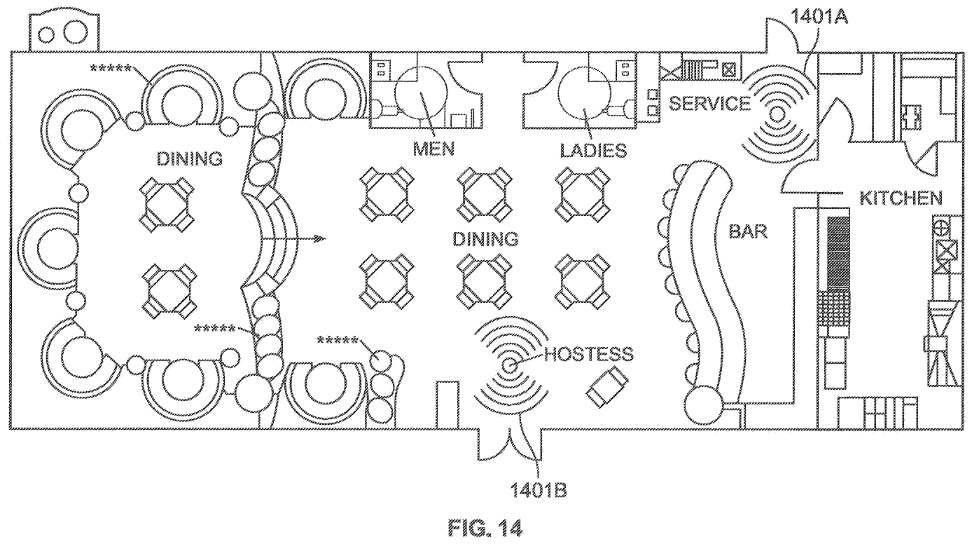

FIGS. 14-18 are diagrams of a CCCN/sensor module layout, according to some embodiments. The layouts apply to different types of Establishments and may identify locations and communications set up. The diagrams show how CCCNs/sensor Modules (SM) may be arranged in various restaurant and store “establishments.” In some embodiments, Establishments would require multiple SM's to cover the ingress/egress points. In some embodiments, increased capability of the sensor module(SM) has also increased flexibility in the method of communication to the cloud. In low volume applications, each SM has the capability of using MQTT to talk directly to the cloud eliminating the need for the on premises CCCN. In some embodiments, in higher volume locations with multiple ingress/egress, the on-premises CCCN manages the large data flows from multiple SM's, synthesizes the activity and sends a condensed information feed to the cloud. The amount of equipment required may be dependent on the floor area/space and the ability for the fewest number of SM's to identify devices and assess entrance and/or exit (presence of those devices).

FIG. 14 is a diagram of a CCCN/sensor module layout, according to some embodiments. FIG. 14 illustrates an Establishment with a single entrance/exit, and potentially an additional exit. Such Establishments may require as few as two 2 SMs (1401A-B) for single entrance/exit layouts. In some embodiments, the goal is to utilize the nominal number of SMs to minimize signal interference, traffic, and data quantity.

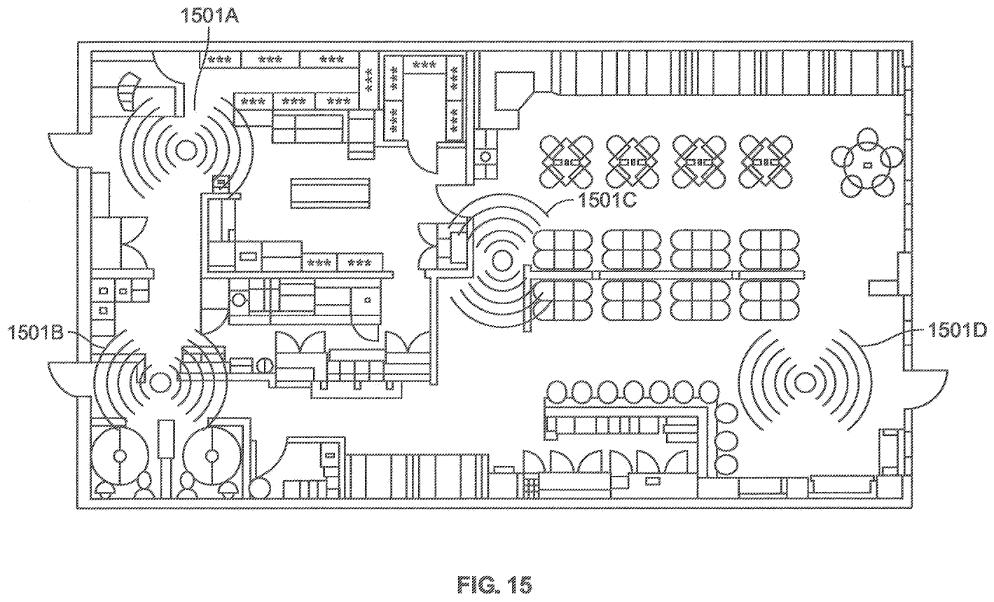

FIG. 15 is a diagram of a CCCN/sensor module layout, according to some embodiments. FIG. 15 illustrates an Establishment with a single entrance/exit, and multiple exits. The Establishment may require unique SM (1501A-D) placement to cover unique entrances and exits and large meeting/dining spaces. In some embodiments, a site survey is required to place sensors properly for highest efficacy and efficiency.

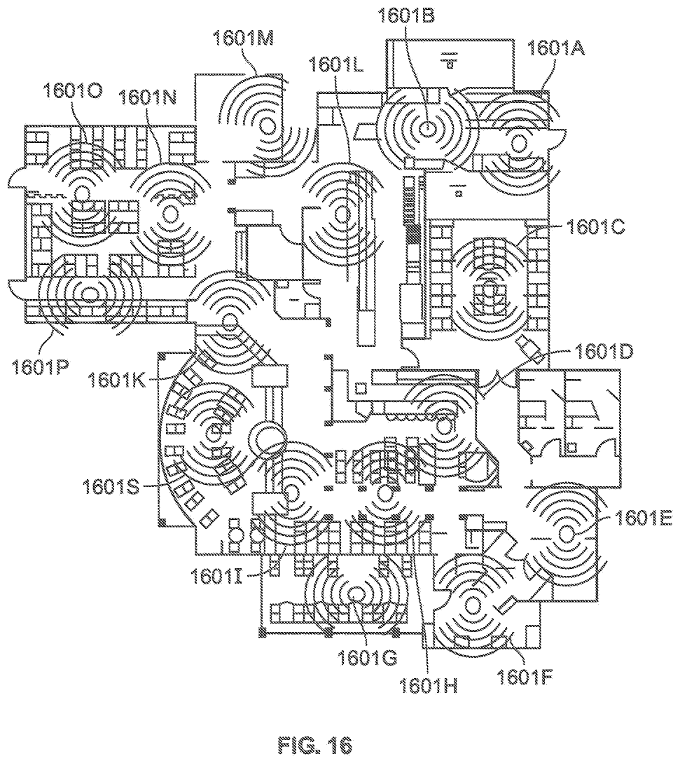

FIG. 16 is a diagram of a CCCN/sensor module layout, according to some embodiments. FIG. 16 illustrates an Establishment with multiple entrance/exits. In some embodiments, a multi-entrance/exit establishment may require as many SMs (1601A-P) to adequately cover passageways, separate rooms, and potential entrances/exits for occupants. The site may be surveyed using temporary SMs to determine the number and location of a final number and location of SMs to provide the minimal, most efficient arrangement for a field deployment. The number of SMs to CCCNs required to manage information may be balanced from a larger number to a smaller number of devices as a result of a site survey. The SM boxes may be multi-directional or unidirectional.

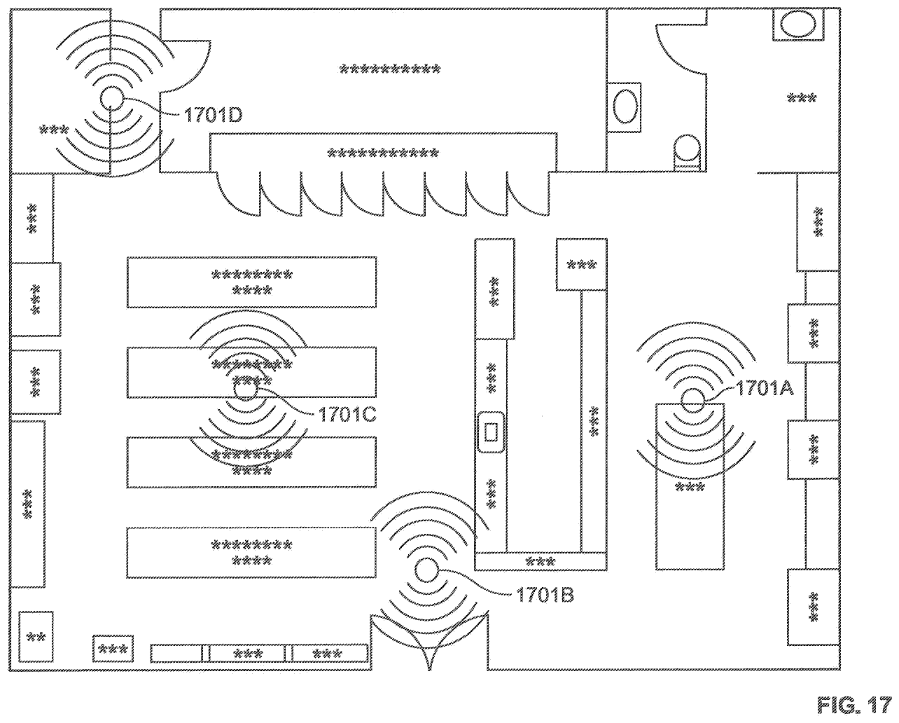

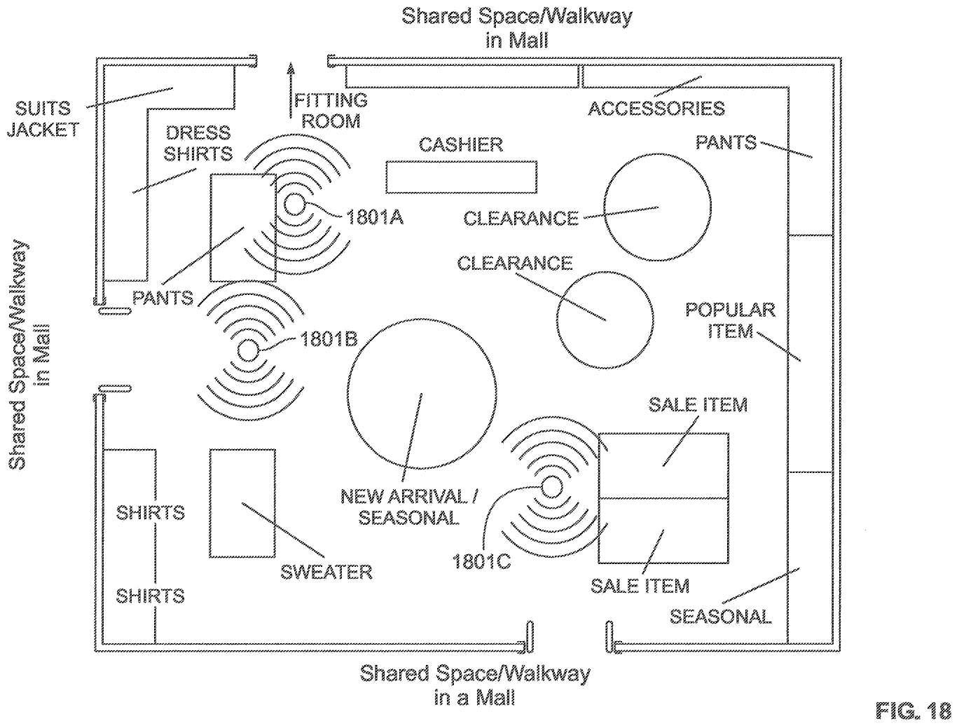

FIG. 17 is a diagram of a CCCN/sensor module layout, according to some embodiments. FIG. 17 illustrates a retail space layout with a discrete entrance and exit. In some embodiments, retail spaces may not require a large number of SMs (1701A-D) due to motion of the occupants.

FIG. 18 is a diagram of a CCCN/sensor module layout, according to some embodiments. FIG. 18 illustrates a retail space with multiple entrance/exit into a shared space, such as a mall setting. Retail/restaurant spaces in strip malls or malls may have outside traffic that may influence CCCNs or sensors (1801A-C) inside the space. CCCNs/sensors will be tailored to account for such transient signals that are not related to occupants of the space.

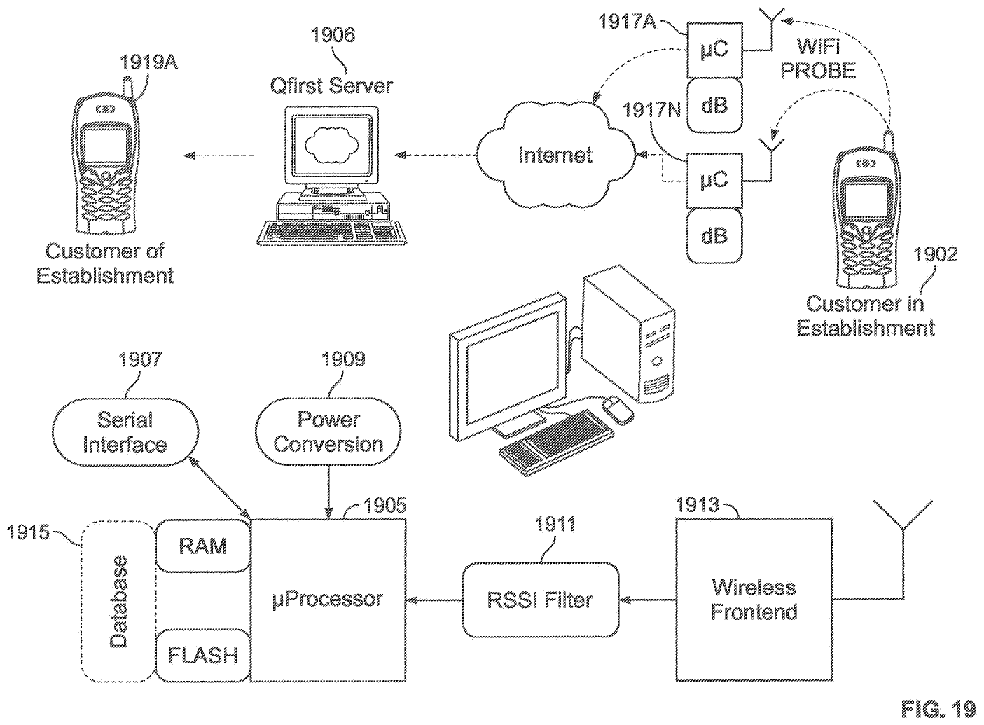

FIG. 19 is an architecture diagram, according to some embodiments. FIG. 19 illustrates a schematic of a CCCN and other components of the system 100.

The CCCN hardware system includes a SM, which may include a system on module (SOM) or system on chip (SOC) that may use a single package 1905 encompassing both a processor and RF components for the sensing devices. The hardware system includes electronics and a serial interface 1907 designed to provide status reports of the “health” of the sensor system for diagnostics and overall sensing systems alterations. The hardware system may include an electronics platform with the appropriate buck or boost capability 1909 to run on either an internal DC battery or via AC. In some embodiments, the hardware system is integrated into a standalone box or appliance that can be plugged into an outlet. In some embodiments, the hardware system can be hard wired into an AC power source. In some embodiments, the hardware system can be integrated into a pass-thru wall socket plug system, or similar device, that has already been U/L certified. In some embodiments, the system may contain a back-up battery to allow for an emergency or set-up power source and which will send a low power/no power signal to the overall system as an alert to elicit repairs if power is lost or the CCCN/sensor malfunctions. The RSSI filter 1911 may be used as discussed above to establish a local region of interest within a given establishment in which devices will be monitored for presence via a wireless frontend 1913. The CCCN may further include memory such as RAM and FLASH, connected with a database 1915.

FIG. 19 further illustrates a flow for a WiFi probe. Probes from customer devices 1902 in the establishment may be picked up wirelessly by one or more sensing devices 1917A-N and transmitted via the Internet to the server 1906. A customer of the establishment 1919A may access one or more applications on the server 1906.

In some embodiments, a CCCN extension system can utilize additional sensing technologies to augment or replace “onsite” capability for establishments using technology other than the basic WiFi/Bluetooth device sensing technology to augment the data used the by QFirst database. For example, in some embodiments, the system utilizes augmenting sensing technologies to assess occupancy even without user engagement with the establishment including but not limited to: (i) back up sensing technologies like radar, sonar, IR, LIDAR, thermal imaging, sound, smell/bio sensing, radio, microwave, millimeter wave, (ii) door open/closing sensors with algorithms to correlate with other electronic sensing systems to assess ingress/egress of occupants, and/or (iii) other “onsite” sensors as needed to augment the occupancy measurement and certainty data when and if cell or Wi-Fi data are interrupted.

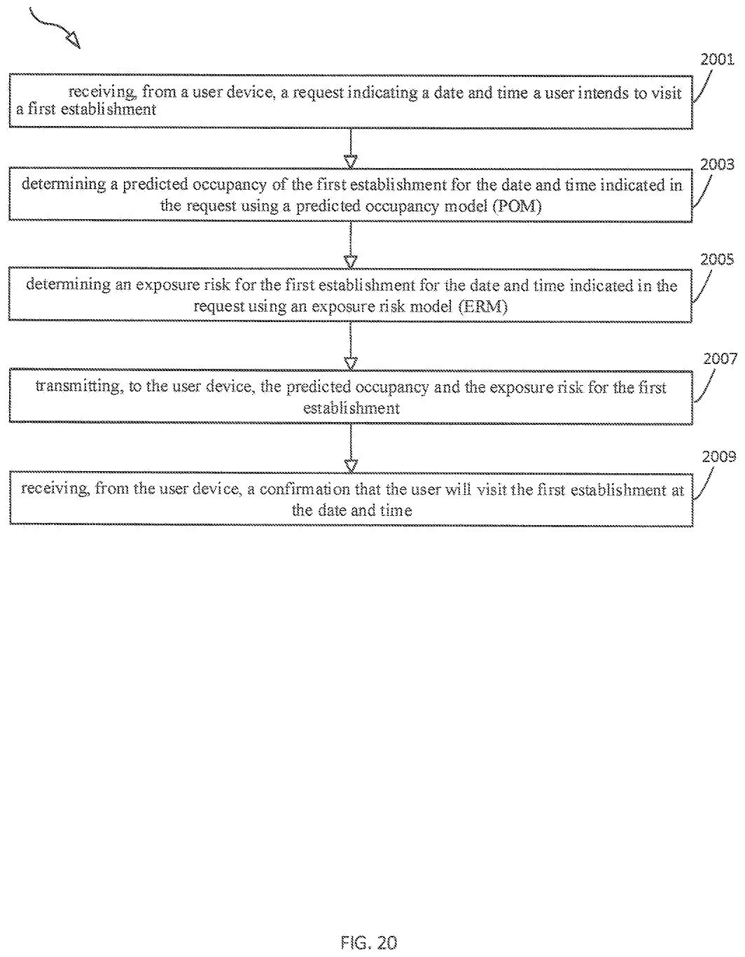

FIG. 20 is a method for providing real-time access, queue, and risk management information, according to some embodiments. In some embodiments, method 2000 is a computer-implemented method for providing real-time access, queue, and risk management information to a user. The method includes step 2001 for receiving, from a user device, a request indicating a date and time a user intends to visit a first establishment. Examples of the screens for generating the request are illustrated in FIGS. 3A-3B, described above. The method includes step 2003 of determining a predicted occupancy of the first establishment for the date and time indicated in the request using a predicted occupancy model (POM). In some embodiments, the POM utilizes one or more of the algorithms described above. The method includes step 2005 for determining an exposure risk for the first establishment for the date and time indicated in the request using an exposure risk model (ERM). In some embodiments, the ERM utilizes one or more of the algorithms described above. The method includes step 2007 for transmitting, to the user device, the predicted occupancy and the exposure risk for the first establishment. The method includes step 2009 for receiving, from the user device, a confirmation that the user will visit the first establishment at the date and time.

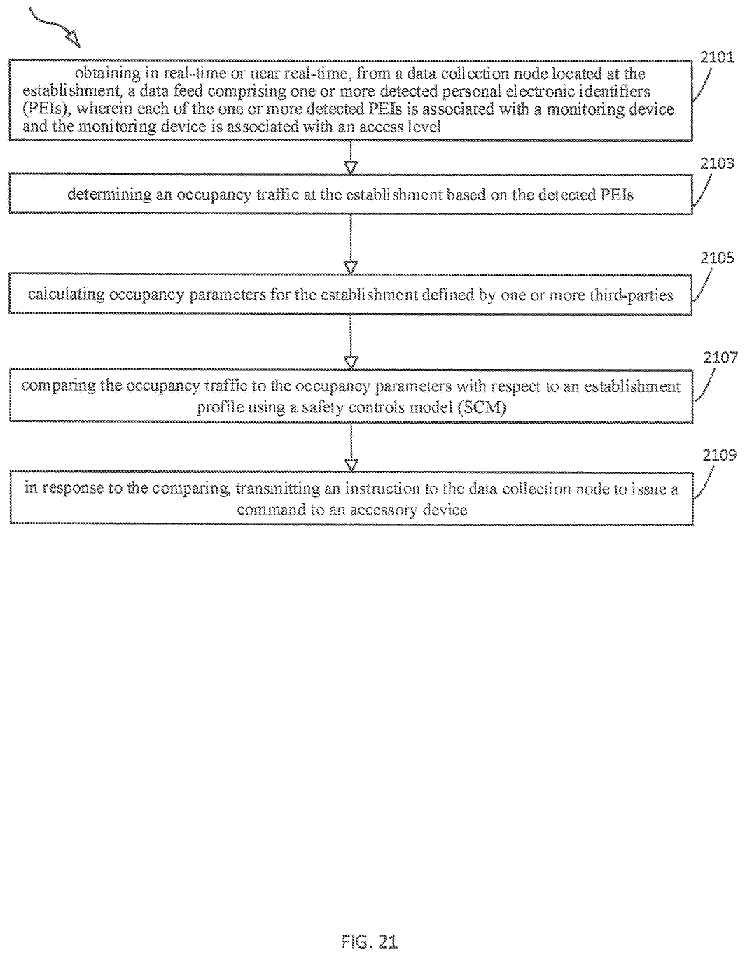

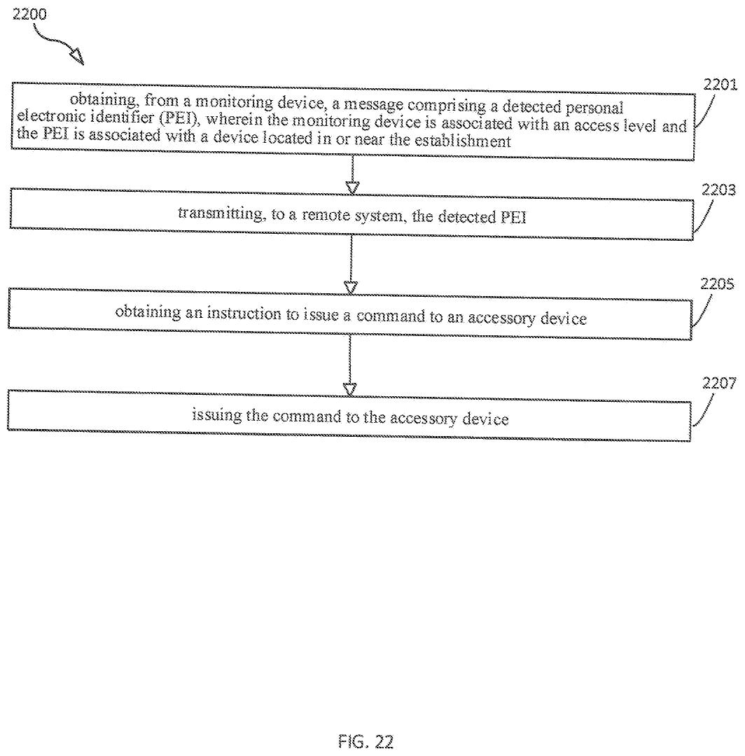

FIG. 21 is a method for providing real-time access, queue, and risk management information, according to some embodiments. In some embodiments, method 2100 is a computer-implemented method for providing real-time access, queue, and risk management information to an establishment. The method includes step 2101 for obtaining (2101) in real-time or near realtime, from a data collection node located at the establishment, a data feed comprising one or more detected personal electronic identifiers (PEIs), wherein each of the one or more detected PEIs is associated with a monitoring device and the monitoring device is associated with an access level. In some embodiments, the data collection node may be an on-premises Computing/Communications/Command Node (CCCN) as discussed above. The method includes step 2103 for determining an occupancy traffic at the establishment based on the detected PEIs. The method includes step 2105 for calculating occupancy parameters for the establishment defined by one or more third-parties. The method includes step 2107 for comparing (2107) the occupancy traffic to the occupancy parameters with respect to an establishment profile using a safety controls model (SCM). The SCM may use one or more of the algorithms described above. The method includes step 2109 for transmitting, in response to the comparing, an instruction to the data collection node to issue a command to an accessory device. In some embodiments, the accessory device may control physical entry doors and limit entry to those who have been granted permission by the system.

FIG. 22 is a method for providing real-time access, queue, and risk management information, according to some embodiments. In some embodiments, 2200 is a computer-implemented method for providing real-time access, queue, and risk management to an establishment. The method includes step 2201 for obtaining, from a monitoring device, a message comprising a detected personal electronic identifier (PEI), wherein the monitoring device is associated with an access level and the PEI is associated with a device located in or near the establishment. The method includes step 2203 for transmitting, to a remote system, the detected PEI. The method includes step 2205 for obtaining an instruction to issue a command to an accessory device. The method includes step 2207 for issuing the command to the accessory device.

The following is a summary of various embodiments described herein:

### AQRM User Embodiments

A1. A method for providing real-time access, queue, and risk management information to a user, the method comprising:
receiving, from a user device, a request indicating a date and time a user intends to visit a first establishment;
determining a predicted occupancy of the first establishment for the date and time indicated in the request using a predicted occupancy model (POM);
determining an exposure risk for the first establishment for the date and time indicated in the request using an exposure risk model (ERM);
transmitting, to the user device, the predicted occupancy and the exposure risk for the first establishment; and
receiving, from the user device, a confirmation that the user will visit the first establishment at the date and time.
A2. The method of embodiment A1, further comprising:
determining, at a predetermined interval prior to the date and time, an updated predicted occupancy of the first establishment; and
transmitting, to the user device, the updated predicted occupancy of the first establishment.
A3. The method of embodiment A1, further comprising:
determining, at a predetermined interval prior to the date and time, an updated exposure risk for the first establishment; and transmitting, to the user device, the updated exposure risk.
A4. The method of embodiment A1, wherein determining the predicted occupancy of the establishment using the POM comprises:
obtaining, from a data collection node located at the first establishment, real-time or near realtime information comprising one or more detected personal electronic identifiers (PEI), wherein each of the one or more PEIs is associated with a person located in or near the first establishment;
obtaining, from a database, historical occupancy traffic associated with the first establishment over a historical time period;
obtaining, from the database, a standard deviation of the historical occupancy traffic; obtaining one or more confirmations that other users will visit the first establishment for the date and time; and,
using the information comprising one or more detected PEIs, the historical occupancy traffic, the standard deviation of the historical occupancy traffic, and the one or more confirmations to determine the predicted occupancy of the first establishment.
A5. The method of embodiment A1, wherein the determining an exposure risk for the first establishment for the date and time indicated in the request using the ERM comprises: obtaining one or more measures of a communicable disease;
obtaining information about the first establishment;
obtaining a duration of the visit at the first establishment at the date and time; and, using the one or more measures, the information about the first establishment, the duration, and the predicted occupancy of the first establishment to determine the exposure risk.
A6. The method of embodiment A5, wherein the one or more measures of a communicable disease comprise one or more of: a percentage of a population infected by the communicable disease, effectiveness of use of personal protection equipment, and usage of personal protection equipment.
A7. The method of embodiment A5, wherein the information about the first establishment comprises one or more of: density of people within the first establishment, expected distancing between people within the first establishment, airflow of the first establishment, and one or more activities performed by persons within the first establishment.
A8. The method of embodiment A1, further comprising:
obtaining, from a data collection node located at the first establishment, first information indicating that the user is physically present at the establishment on a date and time for a time duration;
obtaining, over a predetermined interval of time, second information indicating that the user is physically present at a second establishment on a date and time within the predetermined interval of time for a second time duration;
determining an actual occupancy of the first establishment and the second establishment for the first date, time, and time duration and for the second date, time, and time duration, respectively; determining an exposure risk for the first establishment and the second establishment for the first date, time, and time duration and for the second date, time, and time duration, respectively; determining a cumulative potential exposure risk using a cumulative potential exposure risk model (CPERM) for the user over the predetermined interval of time based on the determined actual occupancy and exposure risk for the first establishment and second establishment; and, transmitting the cumulative potential exposure risk to the user device.
A9. The method of embodiment A1, further comprising:
receiving an indication from the user device to establish a trusted relationship with the first establishment.
A10. The method of embodiment A9, further comprising:
transmitting, to the user device, a notification or incentive to visit the first establishment.
A11. The method of embodiment A1, further comprising:
automatically erasing the request and the confirmation after a predetermined time period.
A12. The method of embodiment A1, further comprising:
displaying, on a graphical user interface of the user device, the predicted occupancy and the exposure risk for the first establishment.
A13. The method of embodiment A1, further comprising:
receiving, from the user device, information indicating a risk tolerance level; and, transmitting, to the user device, an alert when at least one of the predicted occupancy and the exposure risk for the first establishment exceed the risk tolerance level.
A14. An Access, Queue, and Risk Management (AQRM) system, the system comprising:
a processor; and a non-transitory computer-readable medium coupled to the processor, wherein the processor is configured to perform any one of the methods recited in embodiments A1-A13.

AQRM Establishment Embodiments
B1. A method for providing real-time access, queue, and risk management information to an establishment, the method comprising:
obtaining in real-time or near real-time, from a data collection node located at the establishment, a data feed comprising one or more detected personal electronic identifiers (PEIs), wherein each of the one or more detected PEIs is associated with a monitoring device and the monitoring device is associated with an access level;
determining an occupancy traffic at the establishment based on the detected PEIs; calculating occupancy parameters for the establishment defined by one or more third-parties; comparing the occupancy traffic to the occupancy parameters with respect to an establishment profile using a safety controls model (SCM); and,
in response to the comparing, transmitting an instruction to the data collection node to issue a command to an accessory device.
B2. The method of embodiment B1, further comprising:
obtaining at least twenty-five to fifty data feeds from the data collection node, each data feed comprising one or more detected PEIs.
B3. The method of embodiment B1, wherein the establishment profile comprises information indicating a type of the establishment, an occupancy of the establishment, and hours of operation of the establishment.
B4. The method of embodiment B1, wherein the accessory device comprises a storefront display and the command comprises an instruction to display information on the storefront display.
B5. The method of embodiment B1, wherein the accessory device comprises an entry point, and the command comprises an instruction to physically open or close the entry point.
B6. The method of embodiment B5, wherein the entry point comprises one of a door, a gate, or a barrier.
B7. The method of embodiment B1, wherein the data feed is obtained in real-time or near real-time over a Bluetooth, Wi-Fi, or cellular communication channel.
B8. The method of embodiment B1, further comprising:
transmitting, to the data collection node, an update for system software installed on the data collection node.
B9. The method of embodiment B1, further comprising:
transmitting, to a user device associated with the establishment, information on occupancy, forecast demand, and resource planning for the establishment.
B10. The method of embodiment B1, further comprising:
receiving, from a first user device, a request to communicate with one or more trusted visitors of the establishment; and,
in response to the request, transmitting, to one or more user devices associated with the one or more trusted visitors, a notification or incentive to visit the establishment.
B11. An Access, Queue, and Risk Management (AQRM) system, the system comprising:
a processor; and
a non-transitory computer-readable medium coupled to the processor, wherein the processor is configured to perform any one of the methods recited in embodiments B1-B9.

### CCCN Embodiments

C1. A method for providing real-time access, queue, and risk management to an establishment, the method comprising:
obtaining, from a monitoring device, a message comprising a detected personal electronic identifier (PEI), wherein the monitoring device is associated with an access level and the PEI is associated with a person located in or near the establishment;
transmitting, to a remote system, the detected PEI;
obtaining an instruction to issue a command to an accessory device; and issuing the command to the accessory device.
C2. The method of embodiment C1, further comprising:
obtaining, from a plurality of monitoring devices, a plurality of messages comprising a detected PEI.
C3. The method of embodiment C2, wherein the plurality of monitoring devices comprises at least twenty-five to fifty monitoring devices.
C4. The method of embodiment C1, wherein the monitoring device comprises a device configured to capture radio, optical, sound, thermal, or ultrasonic data corresponding to a PEI
C5. The method of embodiment C1, wherein the obtaining the instruction comprises: obtaining, from the remote server, the instruction.
C6. The method of embodiment C1, wherein the message is received over a Bluetooth, Wi-Fi or cellular connection with the monitoring device
C7. The method of embodiment C1, wherein the message further comprises information indicating whether a visitor associated with the detected PEI is entering or exiting the establishment.
C8. The method of embodiment C1, wherein the detected PEI is transmitted over a direct WiFi 6.0 (or future revision protocol) or RJ-45 (or future revision protocol) network connection.
C9. A communications, computing, command node, the node comprising:
a processor; and a non-transitory computer-readable medium coupled to the processor, wherein the processor is configured to perform any one of the methods recited in embodiments C1-C6.
C10. The node of embodiment C9, further comprising:
a battery having a battery life of one year or more.
C11. The node of embodiment C9, further comprising:
a network interface, the network interface configured to connect to a WiFi 6.0 (or future revision protocol) or RJ-45 network (or future revision protocol).
C12. The node of embodiment C9, further comprising:
a sensor array, the sensory array having at least one of radio, ultrasonic, thermal, and optic sensors.

While various embodiments of the present disclosure are described herein, it should be understood that they have been presented by way of example only, and not limitation. Thus, the breadth and scope of the present disclosure should not be limited by any of the above-described embodiments. Generally, all terms used herein are to be interpreted according to their ordinary meaning in the relevant technical field, unless a different meaning is clearly given and/or is implied from the context in which it is used. All references to a/an/the article, element, apparatus, component, layer, means, step, etc. are to be interpreted openly as referring to at least one instance of the article, element, apparatus, component, layer, means, step, etc., unless explicitly stated otherwise. Any combination of the above-described elements in all possible variations thereof is encompassed by the disclosure unless otherwise indicated herein or otherwise clearly contradicted by context.

1. A computer-implemented method for providing real-time access,
queue, and risk management information to a user, the method comprising:
receiving, from a user device, a request indicating a date and time a user intends to visit a first establishment;
determining a predicted occupancy of the first establishment for the date and time indicated in the request using a predicted occupancy model (POM);
determining an exposure risk for the first establishment for the date and time indicated in the request using an exposure risk model (ERM);
transmitting, to the user device, the predicted occupancy and the exposure risk for the first establishment; and
receiving, from the user device, a confirmation that the user will visit the first establishment at the date and time.

2. The method of claim 1, further comprising:
determining, at a predetermined interval prior to the date and time, an updated predicted occupancy of the first establishment; and
transmitting, to the user device, the updated predicted occupancy of the first establishment.

3. The method of claim 1, further comprising:
determining, at a predetermined interval prior to the date and time, an updated exposure risk for the first establishment; and
transmitting, to the user device, the updated exposure risk.

4. The method of claim 1, wherein determining the predicted occupancy of the establishment using the POM comprises:
obtaining, from a data collection node located at the first establishment, real-time or near real-time information comprising one or more detected personal electronic identifiers (PEI), wherein each of the one or more PEIs is associated with a device located in or near the first establishment;
obtaining, from a database, historical occupancy traffic associated with the first establishment over a historical time period;
obtaining, from the database, a standard deviation of the historical occupancy traffic;
obtaining one or more confirmations that other users will visit the first establishment for the date and time; and,
using the information comprising one or more detected PEIs, the historical occupancy traffic, the standard deviation of the historical occupancy traffic, and the one or more confirmations to determine the predicted occupancy of the first establishment.

5. The method of claim 1, wherein the determining an exposure risk for the first establishment for the date and time indicated in the request using the ERM comprises:
obtaining one or more measures of a communicable disease;
obtaining information about the first establishment;
obtaining a duration of the visit at the first establishment at the date and time; and,
using the one or more measures, the information about the first establishment, the duration, and the predicted occupancy of the first establishment to determine the exposure risk.

6. The method of claim 5, wherein the one or more measures of a communicable disease comprise one or more of: a percentage of a population infected by the communicable disease, effectiveness of use of personal protection equipment, and usage of personal protection equipment.

7. The method of claim 5, wherein the information about the first establishment comprises one or more of: density of people within the first establishment, expected distancing between people within the first establishment, airflow of the first establishment, and one or more activities performed by persons within the first establishment.

8. The method of claim 1, further comprising:
obtaining, from a data collection node located at the first establishment, first information indicating that the user is physically present at the establishment on a date and time for a time duration;
obtaining, over a predetermined interval of time, second information indicating that the user is physically present at a second establishment on a date and time within the predetermined interval of time for a second time duration;
determining an actual occupancy of the first establishment and the second establishment for the first date, time, and time duration and for the second date, time, and time duration, respectively;
determining an exposure risk for the first establishment and the second establishment for the first date, time, and time duration and for the second date, time, and time duration, respectively;
determining a cumulative potential exposure risk using a cumulative potential exposure risk model (CPERM) for the user over the predetermined interval of time based on the determined actual occupancy and exposure risk for the first establishment and second establishment; and,
transmitting the cumulative potential exposure risk to the user device.

9. The method of claim 1, further comprising:
displaying, on a graphical user interface of the user device, the predicted occupancy and the exposure risk for the first establishment.

10. The method of claim 1, further comprising:
receiving, from the user device, information indicating a risk tolerance level; and,
transmitting, to the user device, an alert when at least one of the predicted occupancy and the exposure risk for the first establishment exceed the risk tolerance level.

11. The method of claim 1, further comprising:
receiving, from the user device, information relating to a vaccine status or health passport for the user; and
erasing the information relating to a vaccine status or health passport for the user after a predetermined time.

12. An Access, Queue, and Risk Management (AQRM) system, the system comprising:
a processor; and
a non-transitory computer-readable medium coupled to the processor, wherein the processor is configured to:
receive, from a user device, a request indicating a date and time a user intends to visit a first establishment;
determine a predicted occupancy of the first establishment for the date and time indicated in the request using a predicted occupancy model (POM);
determine an exposure risk for the first establishment for the date and time indicated in the request using an exposure risk model (ERM);
transmit, to the user device, the predicted occupancy and the exposure risk for the first establishment; and
receive, from the user device, a confirmation that the user will visit the first establishment at the date and time.

13. A computer-implemented method for providing real-time access, queue, and risk management information to an establishment, the method comprising:
obtaining in real-time or near real-time, from a data collection node located at the establishment, a data feed comprising one or more detected personal electronic identifiers (PEIs), wherein each of the one or more detected PEIs is associated with a monitoring device and the monitoring device is associated with an access level;
determining an occupancy traffic at the establishment based on the detected PEIs;
calculating occupancy parameters for the establishment defined by one or more third-parties;
comparing the occupancy traffic to the occupancy parameters with respect to an establishment profile using a safety controls model (SCM); and,
in response to the comparing, transmitting an instruction to the data collection node to issue a command to an accessory device.

14. The method of claim 13, wherein the establishment profile comprises information indicating a type of the establishment, an occupancy of the establishment, and hours of operation of the establishment.

15. The method of claim 13, wherein the accessory device comprises a storefront display and the command comprises an instruction to display information on the storefront display.

16. The method of claim 13, wherein the accessory device comprises an entry point, and the command comprises an instruction to physically open or close the entry point.

17. The method of claim 16, wherein the entry point comprises one of a door, a gate, or a barrier.

18. The method of claim 13, further comprising:
transmitting, to a device associated with the establishment, information on occupancy, forecast demand, and resource planning for the establishment.

19. The method of claim 13, further comprising:
receiving, from a first user device, a request to communicate with one or more trusted visitors of the establishment; and,
in response to the request, transmitting, to one or more user devices associated with the one or more trusted visitors, a notification or incentive to visit the establishment.

20. An Access, Queue, and Risk Management (AQRM) system, the system comprising:
a processor; and
a non-transitory computer-readable medium coupled to the processor, wherein the processor is configured to:
obtain in real-time or near real-time, from a data collection node located at the establishment, a data feed comprising one or more detected personal electronic identifiers (PEIs), wherein each of the one or more detected PEIs is associated with a monitoring device and the monitoring device is associated with an access level;
determine an occupancy traffic at the establishment based on the detected PEIs;
calculate occupancy parameters for the establishment defined by one or more third-parties;
compare the occupancy traffic to the occupancy parameters with respect to an establishment profile using a safety controls model (SCM); and,
in response to the comparing, transmitting an instruction to the data collection node to issue a command to an accessory device.

21. A computer-implemented method for providing real-time access, queue, and risk management to an establishment, the method comprising:
obtaining, from a monitoring device, a message comprising a detected personal electronic identifier, wherein the monitoring device is associated with an access level and the PEI is associated with a device located in or near the establishment;
transmitting, to a remote system, the detected PEI;
obtaining an instruction to issue a command to an accessory device; and
issuing the command to the accessory device.

22. The method of claim 21, further comprising:
obtaining, from a plurality of monitoring devices, a plurality of messages comprising a detected PEI.

23. The method of claim 21, wherein the monitoring device comprises a device configured to capture radio, optical, sound, thermal, or ultrasonic data corresponding to a PEI.

24. The method of claim 21, wherein the message further comprises information indicating whether a visitor associated with the detected PEI is entering or exiting the establishment.

25. A communications, computing, command node, the node comprising:
a processor; and
a non-transitory computer-readable medium coupled to the processor, wherein the processor is configured to:
obtain, from a monitoring device, a message comprising a detected personal electronic identifier (PEI), wherein the monitoring device is associated with an access level and the PEI is associated with a device located in or near the establishment;
transmit, to a remote system, the detected PEI;
obtain an instruction to issue a command to an accessory device; and
issue the command to the accessory device.

26. The node of claim 25, further comprising:
a sensor array, the sensory array having at least one of radio, ultrasonic, thermal, and optic sensors.
